(19) United States
(12) Patent Application Publication
(10) Pub. No.: US 2025/0143922 A1
(43) Pub. Date: May 8, 2025

(54) SYSTEMS AND METHODS FOR DRUG DELIVERY TO OCULAR TISSUE

(71) Applicant: Regeneron Pharmaceuticals, Inc., Tarrytown, NY (US)

(72) Inventors: Jessica MASTROPIETRO, Schenectady, NY (US); Andrew DUMONT, Rensselaer, NY (US); Bryan GRYGUS, Clifton Park, NY (US); Sarah ELLIS, Albany, NY (US); Parker VALDEZ, Mountain View, CA (US); Trevor LANGLEY, Rensselaer, NY (US); Sindhuja KUCHIBHATLA, Kendall Park, NJ (US); Igor DROBNJAK, Tarrytown, NY (US); Danielle LAIACONA, Troy, NY (US); Daniel HALBIG, Ballston Lake, NY (US); Eric HOLOWKA, Glenmont, NY (US); Bibiana IGLESIAS, Hudson, NY (US); Carmelo ROMANO, Tarrytown, NY (US)

(73) Assignee: Regeneron Pharmaceuticals, Inc., Tarrytown, NY (US)

(21) Appl. No.: 18/941,766

(22) Filed: Nov. 8, 2024

Related U.S. Application Data
(60) Provisional application No. 63/596,998, filed on Nov. 8, 2023.
(60) Provisional application No. 63/600,071, filed on Nov. 17, 2023.

(51) Int. Cl. A61F 9/00; A61M 5/32

(52) U.S. Cl. CPC A61F 9/0008; A61M 5/3243

(57) ABSTRACT

Disclosed are devices and methods for facilitating directed delivery of a medicament into a human organ of a patient. An apparatus to facilitate directed delivery of a medicament into a human organ of a patient may include a container configured to enclose the medicament, a needle having a passage therethrough and a sharp distalmost tip, and a needle guard at least partially surrounding a shaft of the needle. The needle guard may include a distal surface, wherein the sharp distalmost tip of the needle may be configured to extend through an opening in the distal surface. The needle guard may be moveable relative to the needle along an axis of the needle to control a distance from the distal surface to the distalmost tip of the needle.

### CROSS-REFERENCE TO RELATED APPLICATIONS

This application claims priority to U.S. Provisional Patent Application No. 63/596,998, filed on Nov. 8, 2023, and to U.S. Provisional Patent Application No. 63/600,071, filed on Nov. 17, 2023, the entireties of which are incorporated herein by reference.

### TECHNICAL FIELD

Various aspects of the present disclosure relate generally to delivering drugs to ocular tissue. More specifically, the present disclosure relates to instruments and related methods for delivering drugs to, e.g., the suprachoroidal space of an eye.

### INTRODUCTION

Eye conditions and diseases lead to optic nerve damage and visual field loss. Medications, laser surgery, and/or incisional surgery are interventions that may be employed to help lower intraocular pressure, save the subject's existing vision, and delay further progression of the condition and/or disease. With respect to incisional surgery, instruments for performing surgical procedures, devices for delivery drug therapies, and methods made possible by such instruments, are highly sought after to provide improved outcomes for users and subjects.

### SUMMARY OF THE DISCLOSURE

According to an aspect of the disclosure, an apparatus for delivering a medicament to ocular tissue may include: a container configured to enclose the medicament; a needle having a shaft defining a needle axis, a passage therethrough, and a sharp distalmost tip, wherein the passage may be configured to convey the medicament through the needle; and a needle guard at least partially surrounding the shaft of the needle, wherein the needle guard may include a distal surface, wherein the sharp distalmost tip of the needle may be configured to extend through an opening in the distal surface; wherein the needle guard may be moveable relative to the needle along the needle axis to control a distance from the distal surface to the distalmost tip of the needle.

According to another aspect of the disclosure, an apparatus for delivering a medicament to ocular tissue may include: a needle having a shaft defining a needle axis and a sharp distalmost tip and a needle guard at least partially surrounding the shaft of the needle. The needle guard may include a distal surface, wherein the sharp distalmost tip of the needle may be configured to extend through an opening in the distal surface. The needle guard may be moveable relative to the needle along the needle axis. The apparatus may further include a dial coupled to one or more of the needle and the needle guard, wherein rotation of the dial may be configured to alter a distance from the distal surface to the sharp distalmost tip.

In still another aspect of the disclosure, a method of delivering a medicament to ocular tissue using a delivery device including a container configured to enclose the medicament, a needle having a sharp distalmost tip, and a needle guard at least partially surrounding a shaft of the needle, may include: adjusting a distance from a distal surface of the needle guard to the distalmost tip of the needle; inserting, after adjusting the distance, the distalmost tip of the needle into the ocular tissue; positioning the distal surface of the needle guard against an outermost surface of the ocular tissue; and delivering a volume of the medicament to the ocular tissue via the needle.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are several embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1 is a perspective view of an exemplary instrument for treating ocular tissue, according to an embodiment of the present disclosure.

FIG. 2 is a cross-sectional view of an exemplary instrument for treating ocular tissue, according to an embodiment of the present disclosure.

FIG. 3 depicts an exemplary instrument for treating ocular tissue, according to an embodiment of the present disclosure.

FIG. 4 is a cross-sectional view of an exemplary instrument for treating ocular tissue, according to a further embodiment of the present disclosure.

FIG. 5 is a perspective view of an exemplary instrument for treating ocular tissue, according to another embodiment of the present disclosure.

FIGS. 6A and 6B are perspective views of an exemplary instrument for treating ocular tissue, according to yet another embodiment of the present disclosure.

FIG. 7 is a perspective view of an exemplary instrument for treating ocular tissue, according to an embodiment of the present disclosure.

FIG. 8 depicts an exemplary instrument for treating ocular tissue, according to an embodiment of the present disclosure.

FIG. 9 depicts an exemplary instrument for treating ocular tissue, according to an embodiment of the present disclosure.

FIG. 10 depicts an exemplary instrument for treating ocular tissue, according to an embodiment of the present disclosure.

FIGS. 11A and 11B depict an exemplary instrument for treating ocular tissue, according to an embodiment of the present disclosure.

FIG. 12 depicts an exemplary instrument for treating ocular tissue, according to an embodiment of the present disclosure.

FIGS. 13A, 13B, and 13C depict an exemplary instrument for treating ocular tissue, according to an embodiment of the present disclosure.

FIGS. 14A, 14B, and 14C depict relationships of metrics related to the treatment of ocular tissue, according to an embodiment of the present disclosure.

FIGS. 15A, 15B, and 15C depict an exemplary instrument for treating ocular tissue, according to an embodiment of the present disclosure.

FIGS. 16A and 16B depict an exemplary instrument for treating ocular tissue, according to an embodiment of the present disclosure.

FIGS. 17A and 17B depict an exemplary instrument for treating ocular tissue, according to an embodiment of the present disclosure.

FIGS. 18A and 18B depict an exemplary instrument for treating ocular tissue, according to an embodiment of the present disclosure.

FIG. 19 depicts an exemplary instrument for treating ocular tissue, according to an embodiment of the present disclosure.

FIGS. 20A and 20B depict an exemplary instrument for treating ocular tissue, according to an embodiment of the present disclosure.

FIGS. 21A, 21B, and 21C depict exemplary instruments for treating ocular tissue, according to an embodiment of the present disclosure.

FIGS. 22A, 22B, and 22C depict exemplary instruments for treating ocular tissue, according to an embodiment of the present disclosure.

FIG. 23 depicts an exemplary instrument for treating ocular tissue, according to an embodiment of the present disclosure.

FIGS. 24A and 24B depict exemplary instruments for treating ocular tissue, according to an embodiment of the present disclosure.

FIGS. 25A and 25B depict exemplary instruments for treating ocular tissue, according to an embodiment of the present disclosure.

FIGS. 26A and 26B depict exemplary instruments for treating ocular tissue, according to an embodiment of the present disclosure.

FIG. 27 depicts an exemplary instrument for treating ocular tissue, according to an embodiment of the present disclosure.

FIGS. 28A and 28B depict an exemplary instrument for treating ocular tissue, according to an embodiment of the present disclosure.

FIG. 29 depicts an exemplary instrument for treating ocular tissue, according to an embodiment of the present disclosure.

FIG. 30 depicts an exemplary instrument for treating ocular tissue, according to an embodiment of the present disclosure.

FIGS. 31A and 31B depict exemplary instruments for treating ocular tissue, according to an embodiment of the present disclosure.

FIGS. 32A and 32B depict exemplary instruments for treating ocular tissue, according to an embodiment of the present disclosure.

FIG. 33 depicts an exemplary instrument for treating ocular tissue, according to an embodiment of the present disclosure.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art would appreciate that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Aspects of the disclosure relate to, among other things, instruments and methods for delivering drugs to ocular tissues. Each of the aspects disclosed herein may include one or more of the features described in connection with any of the other disclosed aspects. It may be understood that both the foregoing general description and the following detailed description are exemplary and explanatory only and are not restrictive of any claimed inventions.

While this disclosure describes certain instruments and methods, additional descriptions relevant to the instruments and methods described herein may be found in U.S. application Ser. No. 17/444,897, published as US 2022/0047420 A1, and U.S. application Ser. No. 18/336,148, published as US 2023/0405238 A1, the entireties of which are incorporated herein by reference.

The suprachoroidal space (SCS) is a potential space between the sclera and choroid, which traverses the circumference of the posterior segment of the eye. The SCS is a useful site for drug delivery because it targets the choroid, retinal pigment epithelium, and/or retina with high bioavailability, while maintaining low levels elsewhere in the eye. Under normal physiological conditions, primarily due to intraocular pressure (IOP), the SCS is primarily in a collapsed state. The SCS plays a role in maintaining IOP via uveoscleral outflow, which is an alternative drainage route for the aqueous humor, and is a natural flow path from the front to the back of the eye. Due to its role in maintaining IOP, the SCS has the potential to expand and contract in response to the presence of fluid. The SCS may expand to accommodate different volumes, for example, up to about 3.0 mm, depending on injection volumes. Injecting high volumes of drugs may have adverse effects, for example, elevated IOP, retinal elevation, choroidal hemorrhage away from needle entry, and choroidal edema and potential choroidal detachment; backflow from needle entry; and reflux of fluid which may cause subconjunctival hemorrhage. Additionally, high volumes of fluid may not be injected into the eye until a needle of an injection device has fully penetrated the sclera.

To expand the SCS, e.g., by separating the sclera and choroid mechanically and breaking down fibers holding the sclera and choroid together, instruments may be inserted through the sclera and placed at the correct depth between the sclera and choroid layers, such that optimal volumes of fluids, e.g., drugs or other suitable therapeutic agents, may be injected into the SCS. Any drugs inserted into the SCS may allow for direct drug delivery to the posterior section of the eye to specifically target, e.g., the retina and/or macula. The SCS may also be a useful destination for slow-release formulations such as depot drugs. For example, a depot drug inserted into the SCS may be useful for treating portions in the rear of the eye, such as the retina, retinal pigment epithelium (RPE), choroid, or other portions. From within the SCS, the depot drug may effectively target portions of the rear of the eye without impinging on a visual axis of the eye. Instruments and methods for insertion and injection into the eye may only allow for extension into a certain depth of the ocular layers. For example, under physiological conditions, the sclera layer ranges from about 300 μm to about 1100 μm, the SCS has a thickness of about 35 μm, and the choroid layer ranges from about 50 μm to about 300 μm. Depth of insertion of an instrument for drug delivery into the ocular layers may range from about 0.5 mm to about 1.1 mm. However, such a depth of insertion may penetrate and/or impact additional layers of the ocular tissue, e.g., the choroid, retinal pigment epithelium (RPE), and retina. Penetration of such layers should be minimized as much as possible, such that the desired drug may be directed into the targeted area of the eye via a minimally invasive procedure. For example, injection procedures may be performed as an outpatient procedure. Instruments and methods discussed in the present disclosure address the disadvantages described above, and may increase the ability of the SCS to hold and diffuse optimal volumes of drugs, for example, approximately 50 μL to approximately 500 μL.

The example embodiments described herein may be used in the treatment of a variety of conditions, including ocular conditions. For example, embodiments of the present disclosure may be used in the treatment of refractive errors, macular degeneration, cataracts, retinopathy, retinal detachments, glaucoma, amblyopia, strabismus, any other ocular condition, or any other condition suitable for treatment via tissue in the eye.

The description herein and examples are illustrative and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description.

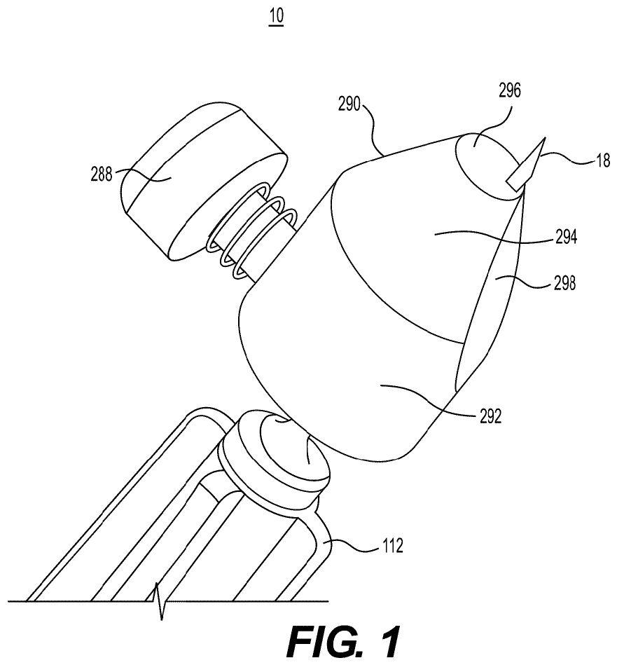

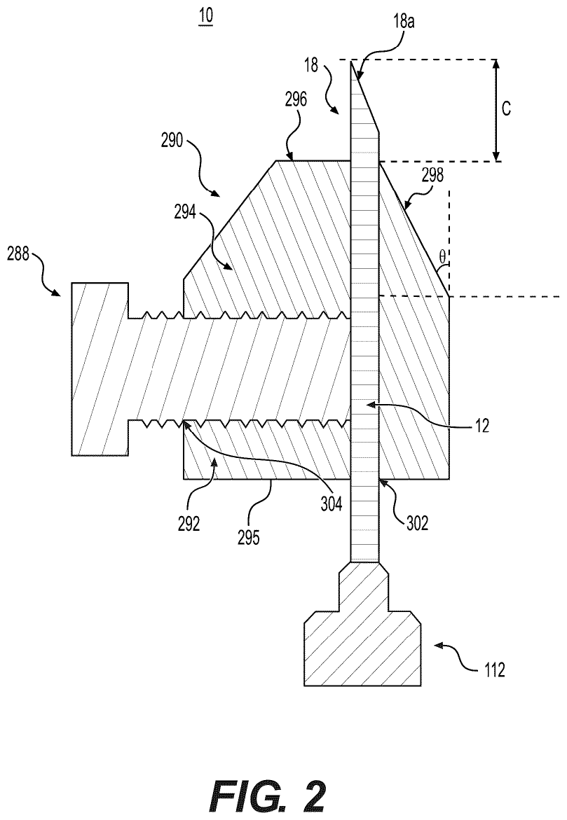

FIGS. 1 and 2 depict an example of an instrument 10 in accordance with the present disclosure. Instrument 10 may include a needle 12 having a passage therethrough configured to serve as a conduit for a medicament. Needle 12 may further include a distalmost tip 18. Distalmost tip 18 may be a sharp tip or needle configured to penetrate a tissue layer of the eye, e.g., a sclera. Needle 12 may be coupled to a needle hub 112, which may further be coupled to a container (not shown). A medicament may be contained within needle 12, needle hub 112, the container, or any combination thereof. In some examples, needle 12 may be a staked needle. In other examples, needle hub 112 may be disposed between the container and needle 12.

Components of instrument 10 may be made of any suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, components of instrument 10 may be made of a rigid material, semi-rigid material, or flexible material, wherein such material may be expandable and/or may allow for various configurations as discussed herein. Materials of instrument 10 may be any biocompatible material that may be sterilized.

Instrument 10 may further include an adaptor 290. Adaptor 290 may be a component configured to surround a shaft of needle 12. Adaptor 290 may be positioned toward distalmost tip 18 relative to needle hub 112 to which needle 12 may be connected. Adaptor 290 may include an intermediate surface 292 which defines a substantially cylindrical portion of adaptor 290. When adaptor 290 is positioned to surround a portion of needle 12, a longitudinal axis of the substantially cylindrical portion of adaptor 290 may extend parallel to a longitudinal axis of needle 12. For purposes of this disclosure, the longitudinal axis of the substantially cylindrical portion should be understood to be a longitudinal axis of adaptor 290.

Adjacent to intermediate surface 292, adaptor 290 may include an angled distal surface 294 disposed toward a distal end of adaptor 290 relative to intermediate surface 292. Angled distal surface 294 may define a substantially frustoconical portion or a partially frustoconical portion of adaptor 290. Angled distal surface 294 may be oriented at an angle ranging from about 30 degrees to about 60 degrees relative to the longitudinal axis of adaptor 290, at an angle ranging from about 40 degrees to about 50 degrees relative to the longitudinal axis of adaptor 290, or at about a 45-degree angle relative to the longitudinal axis of adaptor 290, for example.

Adaptor 290 may further include an outermost slanted surface 298. Outermost slanted surface 298 may be a planar surface adjacent to intermediate surface 292 and/or angled distal surface 294. Alternatively, outermost slanted surface 298 may be a convex surface configured to be placed against and mate with a sclera of a patient's eye. As shown in FIG. 2, outermost slanted surface 298 may be oriented at an angle θ relative to the longitudinal axis of adaptor 290. Angle θ may range from about 25 degrees to about 75 degrees relative to the longitudinal axis of adaptor 290, from about 40 degrees to about 65 degrees relative to the longitudinal axis of the adaptor 290, or from about 30 degrees to about 60 degrees relative to the longitudinal axis of adaptor 290. In an exemplary embodiment, the angle θ may be about 45 degrees relative to the longitudinal axis of adaptor 290.

Outermost slanted surface 298 may be configured in various manners for contact with a sclera of a patient's eye. For example, outermost slanted surface 298 may be smooth or polished to minimize abrasion of the sclera. Alternatively, outermost slanted surface 298 may be rough to minimize movement of adaptor 290 relative to the sclera. In some embodiments, outermost slanted surface 298 may include geometric features, such as protruding dimples, indented dimples, waves, other geometric features, or any combination thereof. Additionally, a coating may be applied to outermost slanted surface 298. The coating may be therapeutic, antibacterial, and/or sterilizing. In some embodiments, a topical anesthetic may be applied as a coating to outermost slanted surface 298. As another example, outermost slanted surface 298 may be formed by overmolding a material on adaptor 290. The overmolded material may be selected, for example, based on its surface properties (e.g., rough, smooth, etc.) or its suitability for surface finishing, such as polishing. Outermost slanted surface 298 may further incorporate various combinations of the aforementioned features, such as a polished surface with geometric features, a rough surface with geometric features, an overmolded material with a coating, etc. While exemplary combinations of features have been described herein, these combinations are not intended to be limiting and other combinations are contemplated.

Adaptor 290 may include visual indications of a position of adaptor 290 and/or of outermost slanted surface 298. For example, outermost slanted surface 298 may be colored differently than other surfaces of adaptor 290 to distinguish outermost slanted surface 298 from the other surfaces. Adaptor 290 may also include visible markings to indicate a position of adaptor 290 and/or of outermost slanted surface 298. Such visible markings may include markings of contrasting color, textured markings, or the like on outermost slanted surface 298 and/or on other surfaces of adaptor 290. The visible markings may be applied to adaptor 290 using silk-screening, overmolding, etching, or various other suitable techniques. The visible markings may be of any geometric shape, including circles, ovals, polygons, irregular shapes, or any combination thereof.

Adaptor 290 may include a proximal surface 295 and a distal surface 296. Proximal surface 295 may be a substantially circular surface adjacent to intermediate surface 292 and existing in a plane perpendicular to the longitudinal axis of adaptor 290. Distal surface 296 may also be a substantially circular surface. Distal surface 296 may be adjacent to angled distal surface 294 and exist in a separate plane perpendicular to the longitudinal axis of adaptor 290. Accordingly, proximal surface 295 may be parallel to distal surface 296.

Adaptor 290 may include a needle bore 302 in which needle 12 may be positioned. Needle bore 302 may extend parallel or substantially parallel to the longitudinal axis of adaptor 290. When positioned in needle bore 302, needle 12 may intersect each of proximal surface 295 and distal surface 296. When positioned in the needle bore, distalmost tip 18 of needle 12 may extend a distance C from distal surface 296. A length of distance C may be such that a bevel 18a of distalmost tip 18 may extend from distal surface 296. The length of distance C may further be such that a portion of a shaft of needle 12 proximal to distalmost tip 18 may extend from distal surface 296. Distance C may be, for example, between 200 μm and 1200 μm, between 400 μm and 1000 μm, between 600 μm and 800 μm, or about 700 μm. In some implementations, bevel 18a and outermost slanted surface 298 may be oriented at the same angle relative to the longitudinal axis of the adaptor 290.

Adaptor 290 may be selectively translatable relative to needle 12 along the longitudinal axis of needle 12. Translation of adaptor 290 may be desirable to adjust the distance C, for example. In some embodiments, the adaptor 290 may be fastened to needle 12. Adaptor 290 may be connected to needle 12 by any suitable means, including by a screw, a fastener, a nut, a bolt, or adhesive. As an example, and as shown in FIGS. 1 and 2, adaptor 290 may be fastened to needle 12 using a screw 288. Screw 288 may be inserted into a threaded bore 304 within adaptor 290. When tightened, screw 288 may exert a force on needle 12 perpendicular to the longitudinal axis of needle 12. The force may result in friction in a longitudinal direction between needle 12 and screw 288, as well as between needle 12 and needle bore 302, thereby preventing adaptor 290 from translating relative to needle 12. If the user wishes to adjust the distance C, e.g., to extend a distance of distalmost tip 18 from distal surface 296, the user may loosen screw 288 to thereby allow translation of adaptor 290 relative to needle 12.

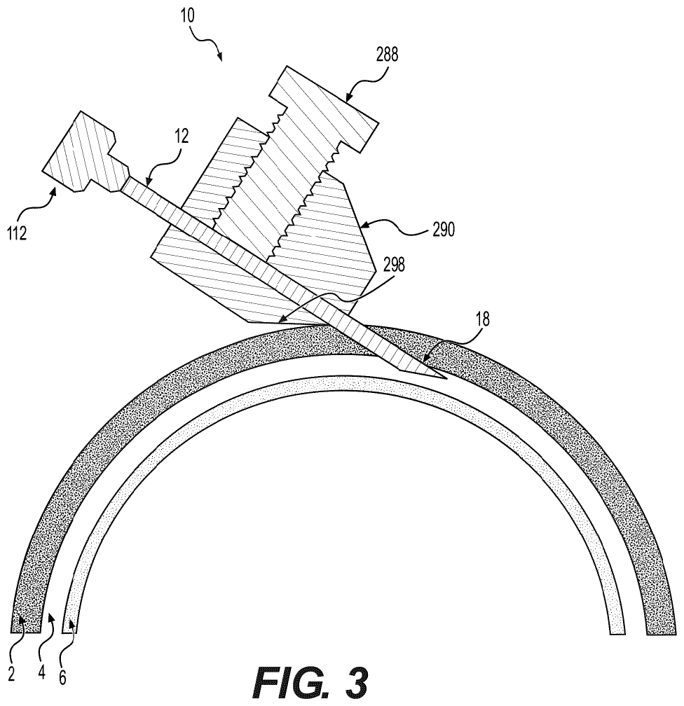

An exemplary use case for instrument 10 is depicted in FIG. 3, in which instrument 10 is shown relative to layers of the eye, i.e., sclera 2, SCS 4, and choroid 6. As shown, adaptor 290 may be used to guide a trajectory of distalmost tip 18 of needle 12 through sclera 2 into SCS 4. To inject a medicament into SCS 4, a user may, for example, penetrate sclera 2 with distalmost tip 18 and insert needle 12 through sclera 2. The user may angle needle 12 such that outermost slanted surface 298 is oriented parallel to a plane tangent to an outer surface of sclera 2. The user may then continue to insert needle 12 until the outermost slanted surface 298 contacts the surface of sclera 2. In an exemplary method in which outermost slanted surface 298 is a planar surface, the user may insert needle 12 until outermost slanted surface 298 is tangent with the surface of sclera 2. In an exemplary method in which outermost slanted surface 298 is a convex surface, the user may insert needle 12 until outermost slanted surface 298 mates with the surface of sclera 2. When outermost slanted surface 298 contacts sclera 2, needle 12 may be prevented from being inserted further and may be prevented from potentially penetrating choroid 6.

In some implementations, the user may be able to adjust the distance C to a desired length by translating adaptor 290 along needle 12. When the user has adjusted distance C and/or angle θ as desired, the user may use adaptor 290 to guide a trajectory of needle 12 into SCS 4 such that it penetrates sclera 2 at a substantially predetermined depth. Thereby, the user may be able to inject the medicament into the suprachoroidal space 4 with relative accuracy without penetrating choroid 6.

As shown in FIGS. 1-3, adaptor 290 may be positioned about needle 12. Adaptor 290 may alternatively be attached to either or both of needle hub 112 and a medicament container (e.g., a syringe) connected to needle 12. In some embodiments, adaptor 290 may be spring-loaded such that a spring urges adaptor 290 toward distalmost tip 18. In use, the user may place adaptor 290 against the patient's sclera, and exert a force sufficient to depress the spring, thereby exposing needle 12. The spring may be configured to control a depth of penetration of needle 12 into the patient's eye.

Adaptor 290 may be made of any suitable material, such as a suitable metal, polymer, and/or combination of metals and/or polymers. Exemplary metallic materials may include stainless steel, nitinol, titanium, and/or alloys of these metals. Exemplary polymeric materials may include polyetheretherketone (PEEK), polyimide, and polyethersulfone (PES). In some examples, adaptor 290 may be made of a rigid material, semi-rigid material, or flexible material. Adaptor 290 may further be formed of any biocompatible material that may be sterilized. In some examples, adaptor 290 may be made of a transparent material to permit easier identification of, and/or navigation relative to, blood vessels in a patient's eye.

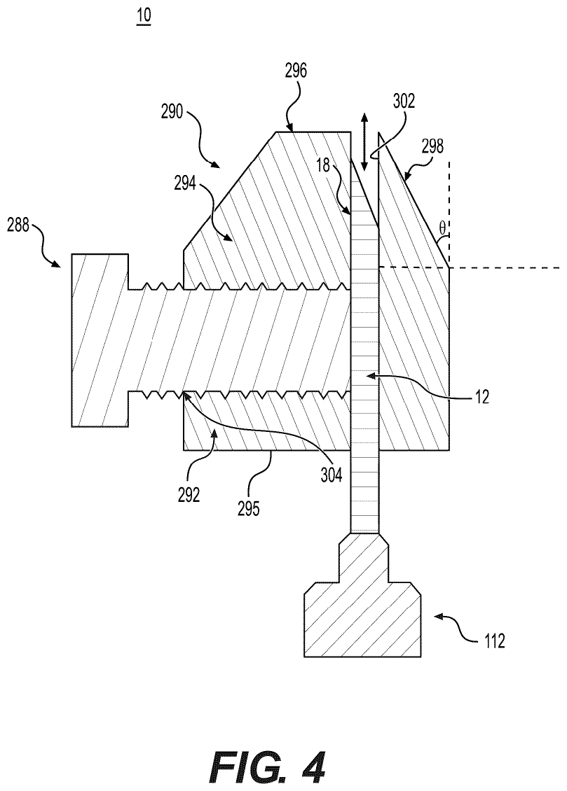

In some embodiments, needle 12 and/or distalmost tip 18 may be retractable. Specifically, distalmost tip 18 may be moveable between a retracted position, in which distalmost tip 18 is positioned within adaptor 290, and a deployed position, in which distalmost tip 18 protrudes from adaptor 290. For example, as shown in FIGS. 4 and 5, prior to use of instrument 10, distalmost tip 18 may be in the retracted position within needle bore 302 of adaptor 290 to prevent inadvertent insertion of distalmost tip 18 or intended injury thereby. During an injection, distalmost tip 18 may be moved to the deployed position, which may be a position similar to the position shown in FIGS. 1, 2, and 3, in which distalmost tip 18 protrudes at least partially out of needle bore 302 past distal surface 296.

Instrument 10 may include a mechanism to move distalmost tip 18 from the retracted position to the deployed position. The mechanism may be of any suitable type, such as a manually powered mechanism, an electrically powered mechanism, a motor driven mechanism, a spring driven mechanism, a compressed gas mechanism, or the like, or any combination thereof. In some embodiments, the mechanism may be user-actuated such that the user may selectively deploy and retract distalmost tip 18. In other embodiments, the deployment of distalmost tip 18 may be user-actuated, but the retraction of distalmost tip 18 may occur automatically at the end of a dose delivery event. In one example, as shown in FIG. 5, the mechanism may include an elastic member 310. Elastic member 310 may be a spring, for example, and may bias needle 12 and/or distalmost tip 18 toward the retracted position. When the user wishes to move distalmost tip 18 from the retracted position to the deployed position, the user may cause elastic member 310 to be depressed, thereby allowing distalmost tip 18 to protrude past distal surface 296. The user may cause elastic member 310 to be depressed using a button, switch, slide, or any other suitable mechanism. In some embodiments, distalmost tip 18 may be configured to remain in the deployed position once moved from the retracted position until the user takes further action to move distalmost tip 18 back to the retracted position. In some embodiments, distalmost tip 18 may be configured to move to the retracted position unless the user continues to actively depress elastic member 310.

In some embodiments, the mechanism that moves distalmost tip 18 from the retracted position to the deployed position may be responsive to signals transmitted by one or more sensors. For example, instrument 10 may include a microprocessor. The microprocessor may be configured to receive signals from one or more sensors, and may further be configured to control the mechanism that moves distalmost tip 18 from the retracted position to the deployed position in response to the signals.

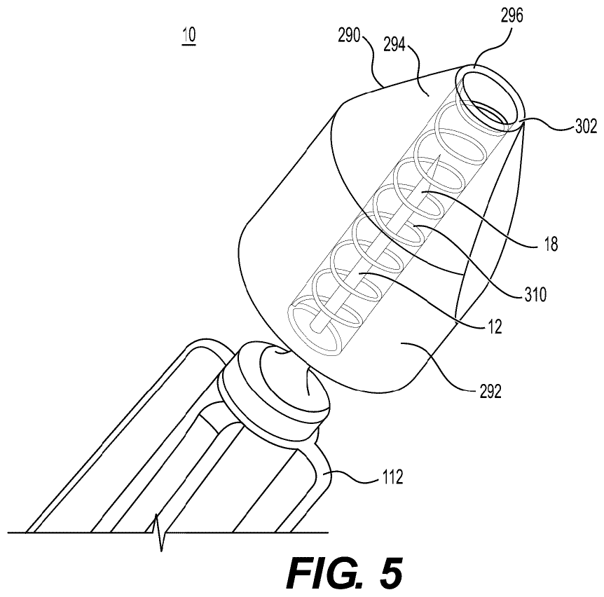

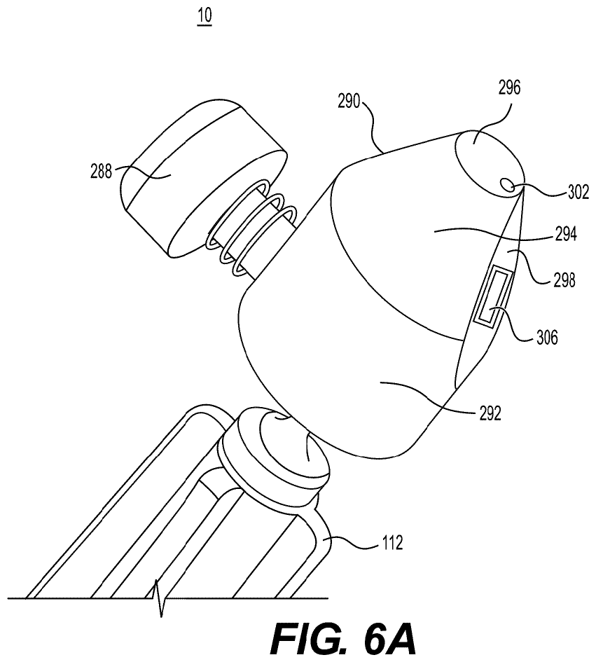

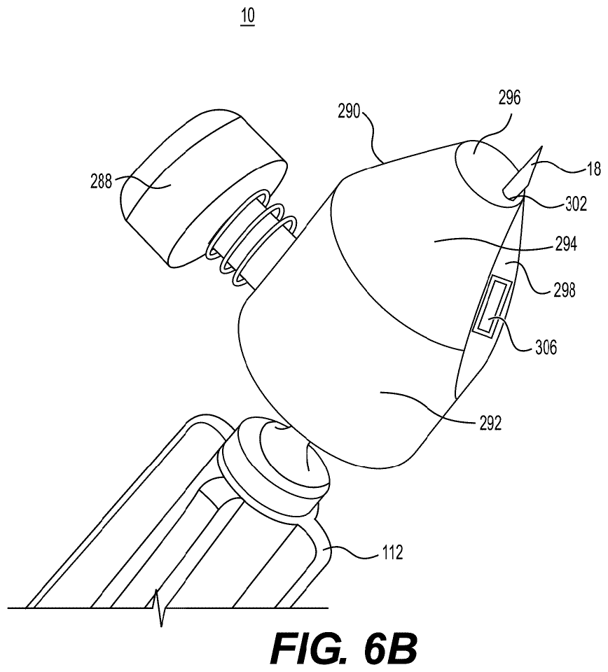

In some embodiments, as shown in FIGS. 6A and 6B, instrument 10 may include a capacitance sensor 306. Capacitance sensor 306 may be positioned on outermost slanted surface 298 of adaptor 290. When capacitance sensor 306 is placed in contact with a sclera of an eye, for example, capacitance sensor 306 may be configured to transmit a signal indicative of contact with the sclera to the microprocessor. In response to receiving the signal, the microprocessor may cause a mechanism to move distalmost tip 18 from the retracted position to the deployed position. In some embodiments, capacitance sensor 306 may be configured to transmit a signal indicative of scleral and choroidal thickness to the microprocessor. In response to the signal, the microprocessor may calculate a distance that the distalmost tip 18 may safely travel forward into the eye. The microprocessor may then cause the mechanism to move distalmost tip 18 forward the calculated distance.

In practice, distalmost tip 18 may initially be in the retracted position prior to an injection, as shown in FIG. 6A. When the user is ready to perform an injection, the user may place outermost slanted surface 298 against the sclera of an eye. Upon placement of outermost slanted surface 298 against the sclera, capacitance sensor 306 may contact the sclera and detect the capacitance thereof. Upon detection of the capacitance of the sclera, capacitance sensor 306 may transmit a signal indicative of contact with the sclera to the microprocessor. In response to receiving the signal, the microprocessor may cause distalmost tip 18 to move from the retracted position to the deployed position, as shown in FIG. 6B. Due to the position of instrument 10 relative to the eye when capacitance sensor 306 detects the sclera, the distalmost tip 18 may penetrate the sclera when moving from the retracted position to the deployed position.

In some embodiments, capacitance sensor 306 may continue to transmit signals to the microprocessor when distalmost tip 18 is in the deployed position. As long as capacitance sensor 306 continues to transmit signals indicating that it remains in contact with the sclera, the mechanism may maintain distalmost tip 18 in the deployed position. If, on the other hand, capacitance sensor 306 is moved out of contact with the sclera, a signal indicating that capacitance sensor 306 is no longer in contact with the sclera may be transmitted to the microprocessor. In response, the microprocessor may cause the mechanism to move distalmost tip 18 to the retracted position.

In some embodiments, the microprocessor may be configured to determine that a drug has been completely administered from instrument 10 or otherwise that a desired amount of a drug has been administered from instrument 10. In response to a determination that the drug has been completely administered or that a desired amount has been administered, the microprocessor may cause the mechanism to move distalmost tip 18 to the retracted position. The microprocessor may initiate such retraction while capacitance sensor 306 remains in contact with the sclera to ensure safe removal of instrument 10 from the patient.

Alternatively, in embodiments in which the mechanism is manually operated, a signal from the capacitance sensor 306 indicative of contact with the sclera may cause one or more visual, audible, or tactile indications to be communicated to the user. For example, upon contact with the sclera, a light on instrument 10 may be illuminated, indicating to the user that instrument 10 is in a suitable position for injection. In another example, a sound may be emitted from instrument 10, indicating to the user that instrument 10 is in a suitable position for injection. In another example, instrument 10 may vibrate, indicating to the user that instrument 10 is in a suitable position for injection. Though examples of visual, audible, and tactile feedback are provided, it should be understood that any suitable indication may be provided to alert the user of a positioning of instrument 10.

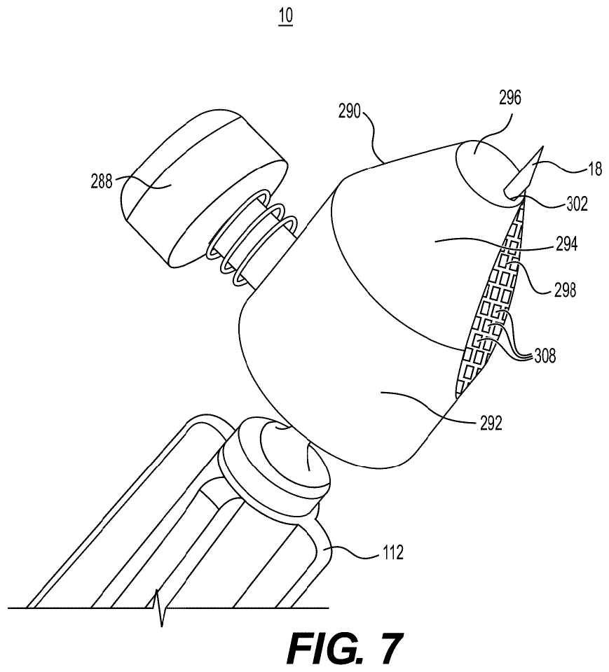

In some embodiments, as shown in FIG. 7, instrument 10 may include one or more pressure sensors 308 (in addition to or as an alternative to capacitance sensor 306). Similar to capacitance sensor 306, pressure sensors 308 may be positioned on outermost slanted surface 298 of adaptor 290. When pressure sensors 308 are placed in contact with a sclera of an eye, for example, each of the pressure sensors 308 may be configured to transmit a signal indicative of a pressure applied by the sclera. In response to receiving signals from the pressure sensors 308 indicative of contact with the sclera, the microprocessor may cause a mechanism to move distalmost tip 18 from the retracted position (not shown) to the deployed position (shown in FIG. 7). In some embodiments, the microprocessor may cause the mechanism to move distalmost tip 18 from the retracted position to the deployed position in response signals indicating a uniform or near-uniform pressure applied across the pressure sensors 308. An indication of a uniform or near-uniform pressure applied across the pressure sensors 308 may signify that outermost slanted surface 298 is uniformly pressed against the sclera, as opposed to positioned at an angle, positioned unfirmly, or the like.

In practice, distalmost tip 18 may initially be in the retracted position prior to an injection. When ready to perform an injection, the user may place outermost slanted surface 298 against the sclera of an eye. Upon placement of outermost slanted surface 298 against the sclera, one or more pressure sensors 308 may contact the sclera and detect the pressure applied to them. Upon detection of pressure indicating contact with the sclera, the one or more pressure sensors 308 may transmit signals indicative of contact with the sclera to the microprocessor. In response to receiving the signals, the microprocessor may cause distalmost tip 18 to move from the retracted position to the deployed position, as shown in FIG. 7. Due to the position of instrument 10 relative to the eye when pressure sensors 308 detect pressures applied by the sclera, the distalmost tip 18 may penetrate the sclera when moving from the retracted position to the deployed position.

In some embodiments, pressure sensors 308 may continue to transmit signals to the microprocessor when distalmost tip 18 is in the deployed position. As long as pressure sensors 308 continue to transmit signals indicative of the sensors being in contact with the sclera, the mechanism may maintain distalmost tip 18 in the deployed position. If, on the other hand, pressure sensors 308 have moved out of contact with the sclera, signals indicating that pressure sensors 308 are no longer in contact with the sclera may be transmitted to the microprocessor. In response, the microprocessor may cause the mechanism to move distalmost tip 18 to the retracted position.

In some embodiments, the microprocessor may be configured to determine that a drug has been completely administered from instrument 10 or otherwise that a desired amount of a drug has been administered from instrument 10. In response to a determination that the drug has been completely administered or that a desired amount has been administered, the microprocessor may cause the mechanism to move distalmost tip 18 to the retracted position. The microprocessor may initiate such retraction while pressure sensors 308 remain in contact with the sclera to ensure safe removal of instrument 10 from the patient.

Alternatively, in embodiments in which the mechanism is manually operated, signals from pressure sensors 308 indicative of contact with the sclera may cause one or more visual, audible, or tactile indications to be communicated to the user, as described herein previously.

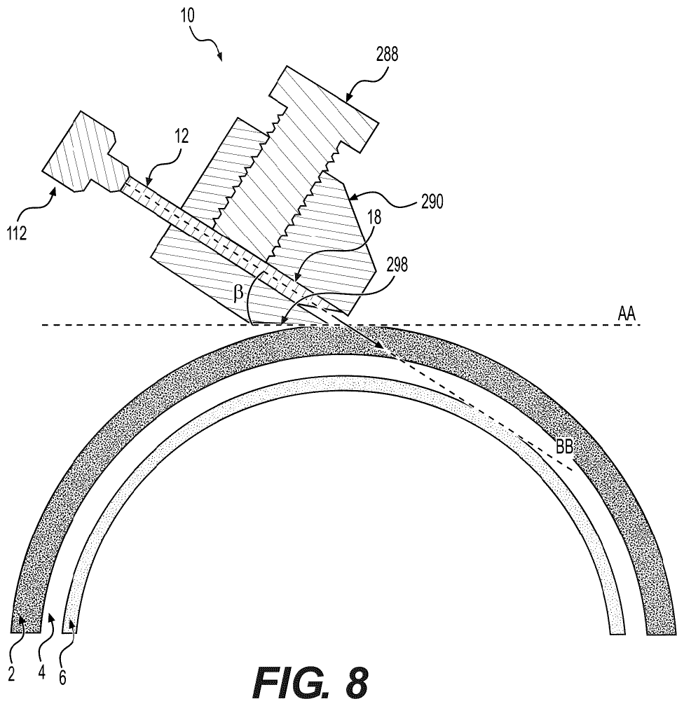

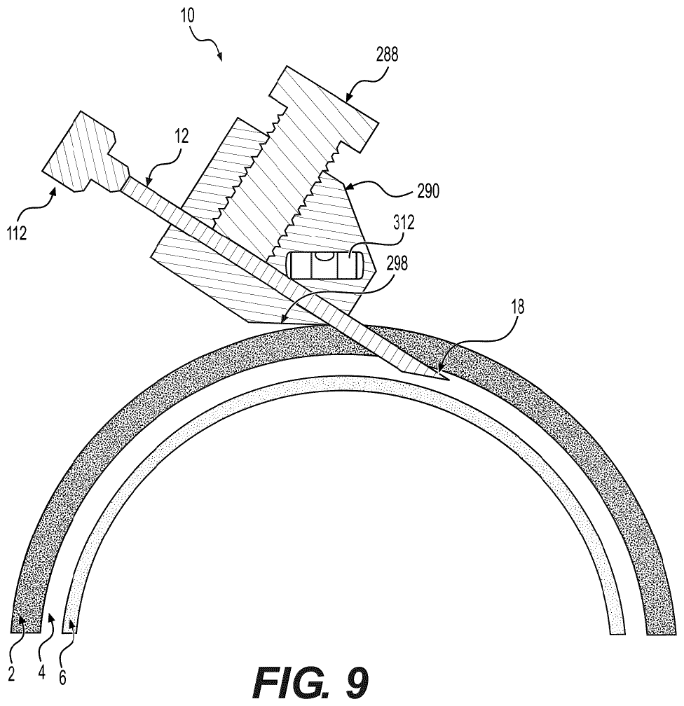

In some embodiments, as shown in FIGS. 8 and 9, instrument 10 may be configured to detect and/or operate according to its angular position. For example, as shown in FIG. 8, instrument 10 may include one or more sensors (e.g., positional or gyroscopic sensors) configured to detect an angle β of an axis BB extending through needle 12 relative to an axis AA extending tangent to sclera 2. The one or more sensors may include image sensors, gyroscopic sensors, accelerometers, combinations thereof, or any other suitable sensors. Each of the sensors may be configured to transmit signals to the microprocessor, which in turn may be configured to calculate angle β based on the signals. In response to a determination that angle β is a suitable angle for injection, the microprocessor may cause the mechanism to move distalmost tip 18 from the retracted position to the deployed position.

In another example, as shown in FIG. 9, instrument 10 may include a level 312 or any other suitable mechanical, electromechanical, or electrical position determining mechanism. In some embodiments, level 312 may be a bubble level, for instance. Level 312 may be angularly offset from needle 12, such that when level 312 is horizontal, needle 12 is at a desired angle relative to horizontal. In use, instrument 10 may be oriented such that level 312 is positioned horizontally (e.g., the bubble is centered). When level 312 is horizontal, needle 12 may be positioned at the desired angle for penetration into the SCS 4. In some embodiments, in response to being placed in a horizontal orientation, level 312 may transmit a signal to the microprocessor indicative of the horizontal orientation. In response to the signal, the microprocessor may cause the mechanism to move distalmost tip 18 from the retracted position to the deployed position.

In some embodiments, the microprocessor may be configured to determine that a drug has been completely administered from instrument 10 or otherwise that a desired amount of a drug has been administered from instrument 10. In response to a determination that the drug has been completely administered or that a desired amount has been administered, the microprocessor may cause the mechanism to move distalmost tip 18 to the retracted position.

The embodiments shown in FIGS. 8 and 9 may alternatively be manually operated. In such embodiments, signals from the sensors and/or level 312 may cause one or more visual, audible, or tactile indications to be communicated to the user, as described herein previously. Subsequently, a user may selectively deploy and retract needle 12 as desired or clinically necessary.

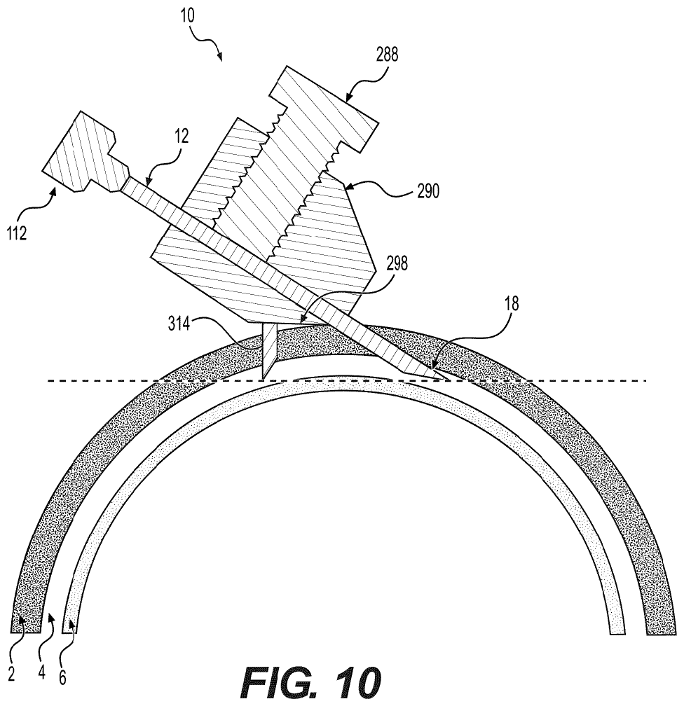

In some embodiments, instrument 10 may be configured to alert the user if distalmost tip 18 has been inserted too deeply into a subject's eye. In such an example, instrument 10 may include a microneedle 314 positioned thereon, as shown in FIG. 10. Microneedle 314 may be positioned in various locations on instrument 10, including on adaptor 290, on needle hub 112, along a shaft of needle 12, on a syringe, or in any other suitable location. In some embodiments, microneedle 314 may be coupled to, for example, outermost slanted surface 298. In such an embodiment, microneedle 314 and needle 12 may both be formed of conductive materials and may be electrically connected to each other on a low voltage circuit. Microneedle 314 may extend a fixed length from the remainder of instrument 10 and may be configured to be inserted to the outermost surface of choroid 6. If distalmost tip 18 is inserted through SCS 4 into choroid 6, an increased electric current may flow through the low voltage circuit. The increased electric current may be detected by the microprocessor and in response to detecting the increased current, the microprocessor may cause one or more visual, audible, or tactile indications to be communicated to the user. The one or more visual, audible, or tactile indications may alert the user that distalmost tip 18 has been inserted too deeply.

In some embodiments, microneedle 314 may be configured to be deployed from and retracted into instrument 10. For example, as described herein previously, capacitance sensor 306 may be configured to transmit a signal indicative of scleral and choroidal thickness to the microprocessor. In response to the signal, the microprocessor may calculate a distance that microneedle 314 may safely travel to reach the outermost surface of choroid 6. The microprocessor may then cause a deployment mechanism to move microneedle 314 the calculated distance into the eye for insertion into the outermost surface of choroid 6.

In some embodiments, in addition to or in lieu of microneedle 314, instrument 10 may include an electrode. In some embodiments, the electrode may be positioned on microneedle 314 and in some embodiments the electrode may be positioned on outermost slanted surface 298. The electrode may be electrically connected to an electrode positioned near distalmost tip 18 on a low voltage circuit. Based on a detected conductivity between the electrodes, the microprocessor may determine whether the electrode on distalmost tip 18 is in contact with sclera 2, is positioned within SCS 4, or is in contact with choroid 6. The microprocessor may be configured to cause one or more visual, audible, or tactile indications to be communicated to the user, where the indications vary depending on the location of distalmost tip 18. The indications may alert the user as to whether distalmost tip 18 has been inserted to a desired depth within the eye (e.g., to the SCS), or whether distalmost tip 18 has been inserted either too shallowly or too deeply.

While instrument 10 is described herein and shown in the associated figures as including a needle 12 that extends through adaptor 290, it should be understood that such a needle is not necessarily required. For example, instrument 10 may instead include a microneedle positioned toward distal surface 296 that does not extend entirely through adaptor 290. In such an embodiment, adaptor 290 may include a fluid conduit therein that may be in fluid communication with the microneedle. Instrument 10 may be configured such that medicament flows through the fluid conduit to the microneedle and into a patient. Such a microneedle may be moveable, as described herein previously, from a retracted position within adaptor 290 to a deployed position in which the microneedle protrudes beyond distal surface 296.

It is to be understood that any dimensions of adaptor 290 perceived from the figures are not intended to be limited and indeed may vary. For example, a length of adaptor 290 (i.e., a distance between proximal surface 295 and distal surface 296) may vary to accommodate needles of different lengths. Also, a diameter of needle bore 302 may vary to accommodate needles having different diameters. Further, diameters of proximal surface 295 and/or distal surface 296 may vary.

As described herein, adaptor 290 may be useful for reducing human error in ocular injection procedures. In addition to being useful for injections into the suprachoroidal space, adaptor 290 may be useful for injections into other spaces in the eye, such as the subretinal space. Current methods for subretinal drug delivery may be invasive and may further require surgery. Surgical procedures for subretinal drug delivery may involve creating tears on the retinal surface and/or full vitrectomies in order to allow for a cannula to access the subretinal space. Alternatively, adaptor 290 may allow access to the subretinal space through the sclera, thereby decreasing the invasiveness of the procedure. Using eye imaging techniques such as optical coherence tomography (OCT) and/or ultrasound, an accurate distance between the surface of the sclera and the subretinal space may be calculated. A distance between distalmost tip 18 of needle 12 and distal surface 296 or outermost slanted surface 298 of adaptor 290 may be configured to match the distance between the sclera and the subretinal space. In such a configuration, adaptor 290 may prevent needle 12 from extending beyond the subretinal space into the vitreous. Outermost slanted surface 298 may also control an angle at which the subretinal injection is performed.

Adaptor 290 may be formed by any suitable manufacturing process, including but not limited to milling, CNC machining, polymer casting, rotational molding, vacuum forming, injection molding, extrusion, blow molding, or any combination thereof.

The various devices and components described herein may be provided in a kit for practicing one or more of the methods described herein. For example, a syringe, a needle, an adaptor, and an amount of ophthalmic drug may be provided in a blister pack. Each of the syringe, the needle, the adaptor, and the ophthalmic drug may be sealed within the blister pack after being sterilized. In some embodiments, a kit may include multiple adaptors. The multiple adaptors may have varying dimensions such that a user may select an adaptor best suited to a patient's anatomy and/or to control a penetration angle or depth of the needle. The multiple adaptors may also be formed from varying materials such that a user may choose an adaptor having an appropriate material for a particular procedure and/or patient. In some embodiments, the syringe may contain the ophthalmic drug. A nominal maximum fill volume of the syringe may be between about 0.5 mL and about 1.0 mL. In various methods described herein, a volume of the medicament, e.g., an ophthalmic drug, delivered to the patient may range from about 50 μL to about 500 μL.

Various drugs and formulations of drugs may be used with the embodiments of the present disclosure. As one example, embodiments described herein may be used to inject a drug in delayed-release pellet form. The drug may be released from the pellets when the pellets are hydrated, which may be achieved either by exposure of the pellets to fluids of the eye, by injecting a separate hydrating fluid, or by a combination of the foregoing. The separate hydrating fluid, such as saline, may be injected either before, after, or simultaneously with the pellets. As another example, embodiments described herein may be used to inject multiple substances in sequence. A first substance may be injected to expand a target space of the eye, such as the suprachoroidal space, and a second substance may subsequently be injected into the expanded suprachoroidal space. The first substance may be, for example, saline and the second substance may be, for example, a drug in a viscous gel form. As still another example, a sponge-like material may first be injected or inserted into a target space of the eye. The sponge-like material may be configured to release a drug over time. The sponge-like material may further be refilled or re-soaked with the drug by subsequent injections of the drug.

Drugs that may be used with embodiments of the present disclosure include: aflibercept (EYLEA®), triamcinolone acetonide suspension (ZUPRATA®), bevacizumab (AVASTIN®), and gene therapy drugs (including adeno-associated virus serotype 8 (AAV8) vectors for ocular gene transfer). Though examples are provided herein, these examples are not intended to be limiting and any suitable drug may be used with the embodiments of the present disclosure.

In embodiments of the present disclosure, needle 12 may be a first needle and the devices, apparatus, and/or kits disclosed herein may include a second needle. The first needle and the second needle may be interchangeable. Accordingly, needle 12 maybe be replaceable.

Embodiments of the present disclosure may further include variations of the instruments previously described and/or additional instruments for treating ocular tissue and/or delivery a drug to ocular tissue. For example, the previously described instruments may further incorporate, and/or be modified according to, the following aspects. Alternatively, the exemplary instruments that follow may be considered separately from the previously described instruments. It is understood that various combinations of the structures, components, and/or elements described herein are contemplated and are within the scope of the present disclosure.

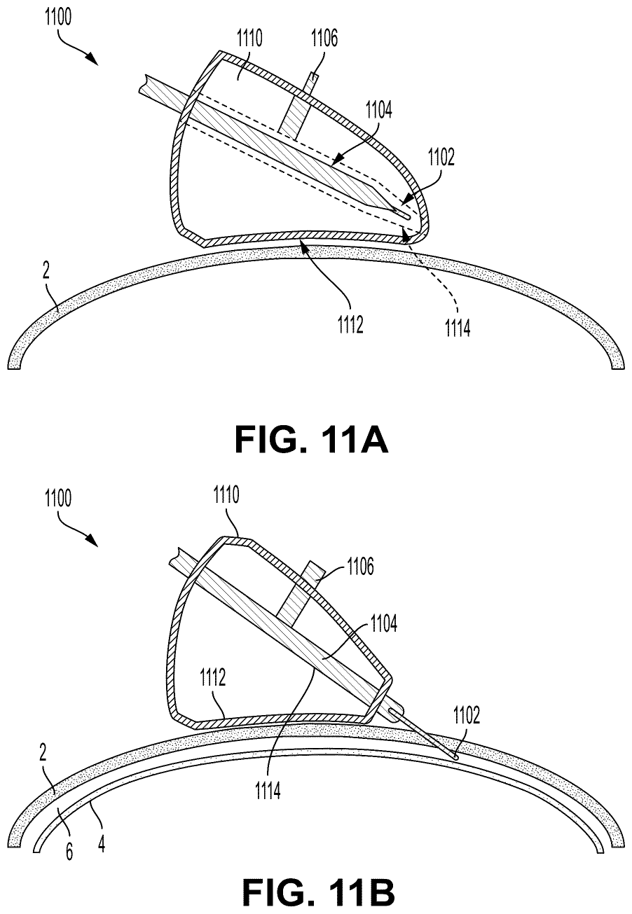

According to FIGS. 11A and 11B, an exemplary drug delivery device 1100 for delivering a drug to ocular tissue may include a needle 1102 attached to a syringe 1104. Syringe 1104 may be positioned within a channel 1114 of a sleeve 1110. Similar to adaptor 290, sleeve 1110 may be configured to surround the syringe 1104 and/or the needle 1102. Syringe 1104 and needle 1102 may be coupled to the sleeve 1110 via a lock 1106. The lock 1106 may be selectively adjustable by a user so as to maintain syringe 1104 and needle 1102 in position with respect to the sleeve 1110, or release syringe 1104 and needle 1102 to allow movement relative to sleeve 1110, as desired. In some embodiments, lock 1106 may be a screw that extends from an outer surface of sleeve 1110 into channel 1114, and the screw may be tightened or loosened to hold or release, respectively, the syringe 1104 and needle 1102, as desired. FIG. 11A shows needle 1102 in a retracted configuration, whereas FIG. 11B shows needle 1102 in a deployed configuration.

Sleeve 1110 may include a curved surface 1112 configured to be placed against sclera 2 of a patient's eye. Sleeve 1110 and curved surface 1112 may be configured to control a depth and angle of insertion of needle 1102 into the patient's eye. In some embodiments, channel 1114 may be inclined (i.e., angled) relative to curved surface 1112, such that needle 1102 is caused to enter a patient's eye at a desirable angle upon insertion and facilitate positioning in the SCS 6. Additionally, in some embodiments, sleeve 1110 may define a maximum distance that needle 1102 may be inserted into the patient's eye. For example, sleeve 1110 may be configured to allow needle 1102 to penetrate only sclera 2 of a patient's eye, but not choroid 4, as shown in FIG. 11B.

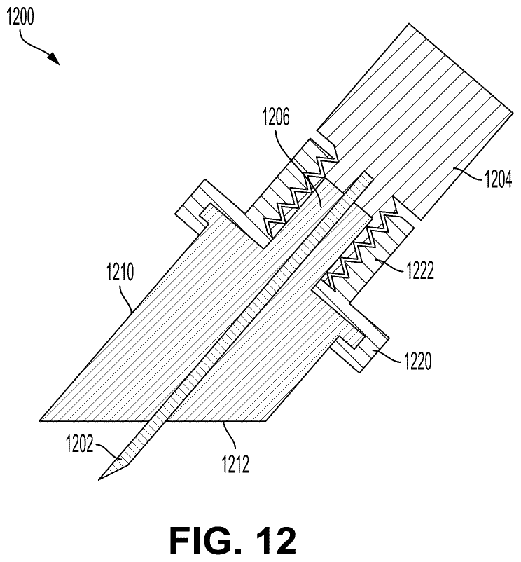

FIG. 12 depicts an exemplary drug delivery device 1200. Drug delivery device 1200 may include a needle 1202 attached to a syringe 1204, and an adaptor 1210 surrounding needle 1202. Similar to adaptor 290, adaptor 1210 may be configured to surround needle 1202 and may include an angled distal surface 1212 for placement on a sclera. Adaptor 1210 may include a stem 1206 that is keyed to syringe 1204 so as to prevent rotation of adaptor 1210 relative to syringe 1204. Needle 1202 may also have a fixed position relative to syringe 1204. Drug delivery device 1200 may further include a dial 1222 having a threaded connection to either or both of syringe 1204 and stem 1206. Upon rotation of dial 1222, adaptor 1210 may be configured to move along an axis of needle 1202, thereby increasing or decreasing a distance that needle 1202 protrudes from adaptor 1210. Accordingly, to achieve a desired needle insertion depth, a user may rotate dial 1222 to expose a corresponding length of needle 1202.

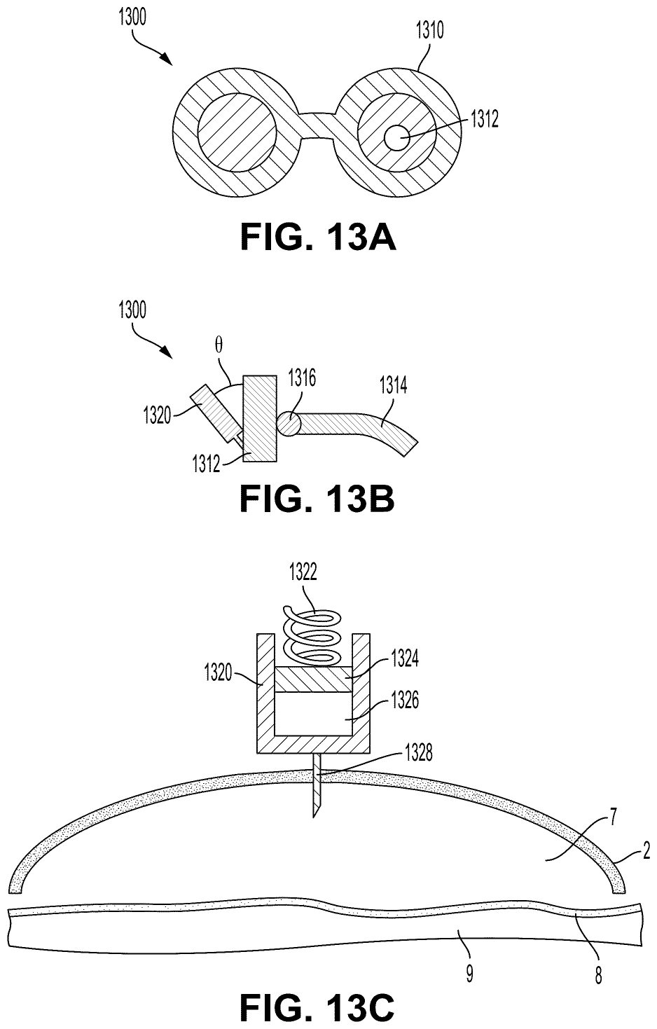

FIGS. 13A, 13B, and 13C illustrate another exemplary drug delivery device 1300 for delivering a drug to ocular tissue. Drug delivery device 1300 may be in the form of a pair of wearable glasses or a mask 1310 configured to be fitted to a patient's face. Drug delivery device 1300 may include a port 1312 configured to be positioned near an eye of a patient and through which a syringe 1320 having a needle may be inserted. Port 1312 may be angled relative to an outer surface of mask 1310 by an angle θ so as to direct the needle into a patient's eye at a desirable angle. Drug delivery device 1300 may include one or more earpieces 1314 attached to lateral sides by a hinge 1316 so that the device may be supported by a patient's ears and may be adjustable to accommodate differing patient anatomies.

In some embodiments, syringe 1320 may include a plunger 1324, which may be driven by a spring 1322, as shown in FIG. 13C. In other embodiments, plunger 1324 may be driven by a motor or any other suitable biasing mechanism. Plunger 1324 may move in an axial direction through the syringe 1320 to force a drug through needle 1328. In some embodiments, a pressure sensor may be included to detect a pressure within syringe 1320 and the detected pressure may be used to determine a desired depth of insertion of needle 1328. For example, a region 7 between the sclera 2 and a retinal pigment epithelium (RPE) 8, which includes the SCS, may have a pressure P₁. A region 9 beneath the RPE 8 may have a pressure P₂, which is greater than P₁. In order to inject a drug into region 7, a pressure sensor may be used to detect a pressure P₃ within the syringe during dispersal of a drug. If P₃ is a value between P₁ and P₂, P₃ may indicate that needle 1328 has penetrated sclera 2 but has not penetrated RPE 8.

Additionally, in some embodiments, a force sensor may be included to detect a force applied to advance needle 1328 into an eye. For example, peaks in detected force as a function of distance may be indicative of penetration of layers of an eye, such as a sclera, choroid, or RPE, which exist respectively at different distances from an outer surface of an eye. In some embodiments, a pressure sensor may be used in lieu of or in addition to a force sensor. In some embodiments, portions of the device responsible for pressure and/or force sensing may be reusable.

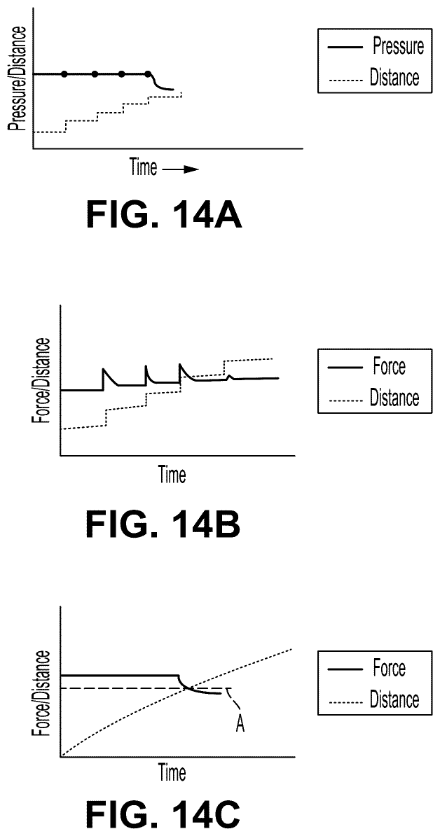

FIGS. 14A, 14B, and 14C depict several charts further illustrating relationships between pressure, force, and distance as a function of time as a needle is inserted into an eye. FIGS. 14A, 14B, and 14C are therefore instructive for incorporating a pressure sensor and/or force sensor in devices described herein. In FIG. 14A, fluid pressure within a syringe and distance of insertion of a needle into an eye are plotted as step-wise functions of time as the needle is advanced into the eye. In some embodiments, the needle may be advanced in small, discrete distances as the measurements are taken. As shown in FIG. 14A, pressure within the syringe may remain stable as the needle advances through one or more layers of the eye. The pressure may then decrease when the needle passes into a space such as the SCS. To avoid inadvertently penetrating further into the eye, the device may cease advancing the needle upon detecting the decrease in pressure and/or may alert the user to the decrease in pressure. Pressure measurements may further be used to trigger end-of-dose actions and/or automatic retraction of the needle from the eye.

FIG. 14B depicts a relation between force applied to a needle and distance of insertion of the needle plotted as step-wise functions of time as the needle is advanced into the eye. In some embodiments, the needle may be advanced in small, discrete distances as the measurements are taken. As shown, the force applied to the needle may spike each time the needle is advanced. Larger force peaks may indicate that the needle is positioned within a layer of the eye, such as the sclera, whereas a lower force peak or a flatting of the force may indicate that the needle has passed through one or more layers into a space such as the SCS. The device may cease advancing the needle and/or may alert the user once a peak force decreases below a predetermined threshold. Force measurements may further be used to trigger end of dose actions and/or automatic retraction of the needle from the eye.

In some embodiments, continuous feedback may be used in lieu of step-wise feedback. FIG. 14C depicts a chart in which force and distance are plotted as continuous functions of time and shows how a force threshold A may be implemented to achieve a desired depth of insertion. For example, upon the insertion force decreasing below a certain threshold value, the device may be configured to automatically cease advancing the needle. The threshold may be calibrated to indicate that the distal end of the needle has reached a desired depth, such as to the SCS, for example. Alternatively, or in addition, a change in the rate of speed of the needle (i.e., acceleration) may be used. For example, acceleration of the needle may indicate that the needle has passed through a layer of the eye into a space such as the SCS and may trigger the device to cease advancing the needle.

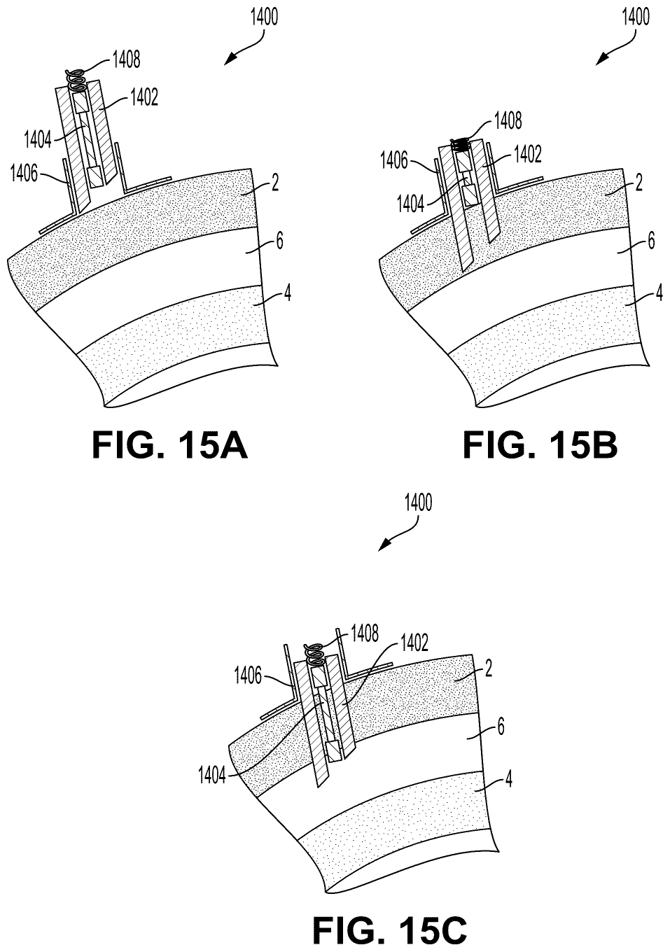

FIGS. 15A, 15B, and 15C depict a drug delivery device 1400 for delivering a drug to ocular tissue. Drug delivery device 1400 may include a needle 1402, a bobbin 1404, an adaptor 1406, and a spring 1408. Bobbin 1404 may be positioned within needle 1402, and bobbin 1404 may move between a first position in which a pathway through needle 1402 is unobstructed and a second position in which the pathway is obstructed. When bobbin 1404 is in the first position, a drug may flow through needle 1402. Bobbin 1404 may be biased by spring 1408 toward the first position in which the pathway is unobstructed.

At ambient pressure, spring 1408 may cause bobbin 1404 to be in the first position, as shown in FIG. 15A. As the needle 1402 is inserted into an eye, an increased pressure may cause the spring 1408 and/or bobbin 1404 to compress and bobbin 1404 to move into the second position, as shown in FIG. 15B. Spring 1408 may be calibrated such that an increased pressure of the sclera 2 causes the bobbin 1404 to move into the second position, thereby preventing dispersal of the drug into the sclera 2. As needle 1402 moves toward lower pressure, such as into the SCS 6 as shown in FIG. 15C, the bobbin 1404 may return to the first position in which the drug may flow through needle 1402. If needle 1402 is advanced further into another layer such as the choroid 4, increased pressure may force bobbin 1404 into the second position, thereby preventing dispersal of drug into the choroid 4.

In some embodiments, a motor or other similar actuator (e.g., a solenoid) may be used to advance needle 1402 and/or drive a plunger within a syringe. A resistance and/or current of the motor may be monitored as needle 1402 is advanced and/or the plunger is driven. Higher current may indicate that needle 1402 faces increased resistance to advancement and may cause the device to cease advancing needle 1402. Similarly, higher current may indicate that a fluid pathway from the syringe is obstructed, indicating that needle 1402 is not positioned within a desirable space for dispersing the drug, such as the SCS.

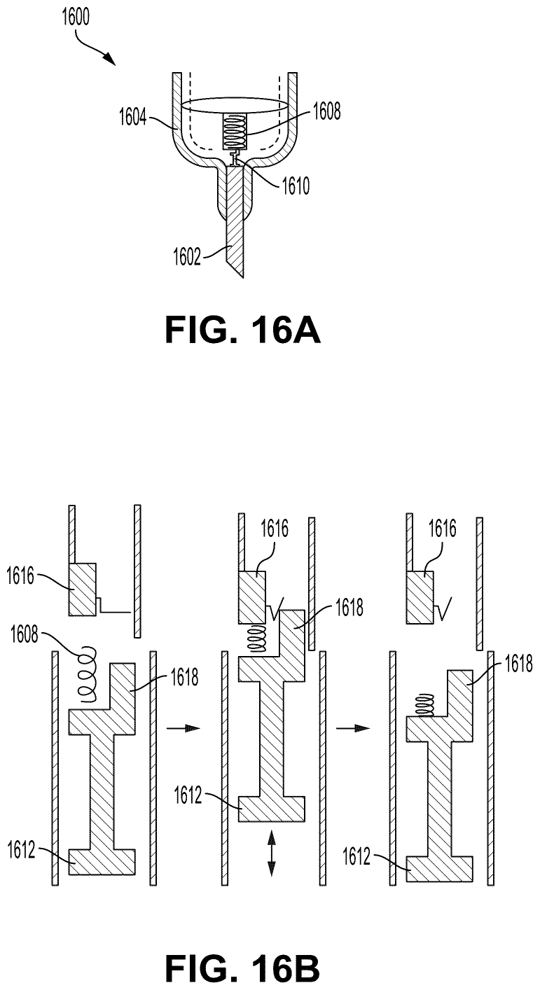

FIGS. 16A and 16B depict a similar drug delivery device 1600 for delivering a drug to ocular tissue. Drug delivery device 1600 may include a needle 1602, a syringe 1604, a flow control mechanism 1610, and a biasing mechanism 1608. Syringe 1604 may be configured to contain a liquid drug, and flow control mechanism 1610 may be configured to control the conditions in which the drug is permitted to flow through needle 1602.

An exemplary configuration of flow control mechanism 1610 is shown in FIG. 16B. Flow control mechanism 1610 may include a bobbin 1612 that is biased toward a first positon by biasing mechanism 1608. Depending on a pressure applied to bobbin 1612, bobbin 1612 may seal or open a fluid pathway from syringe 1604 to needle 1602. As shown in FIG. 16B, a fluid pathway may initially be closed by a valve 1616 and bobbin 1612 may initially be biased away from valve 1616 by biasing mechanism 1608. As needle 1602 is inserted into an eye and a pressure applied to bobbin 1612 increases, bobbin 1612 may be forced upward such that biasing mechanism 1608 becomes compressed, and projection 1618 moves valve 1616 into an open state. As needle 1602 advances further into a lower pressure region of the eye, such as the SCS, bobbin 1612 may move downwardly away from valve 1616 due to force applied by biasing mechanism 1608. When valve 1616 is in the open state and bobbin 1612 is in the downward position, the liquid drug may be permitted to flow from syringe 1604 through needle 1602. In some embodiments, movement of the bobbin 1612 may cause a visual indication to a user. For example, when needle 1602 is inserted to a desired depth within an eye, bobbin 1612 may cause a corresponding visual indication to the user indicating that drug delivery should proceed.

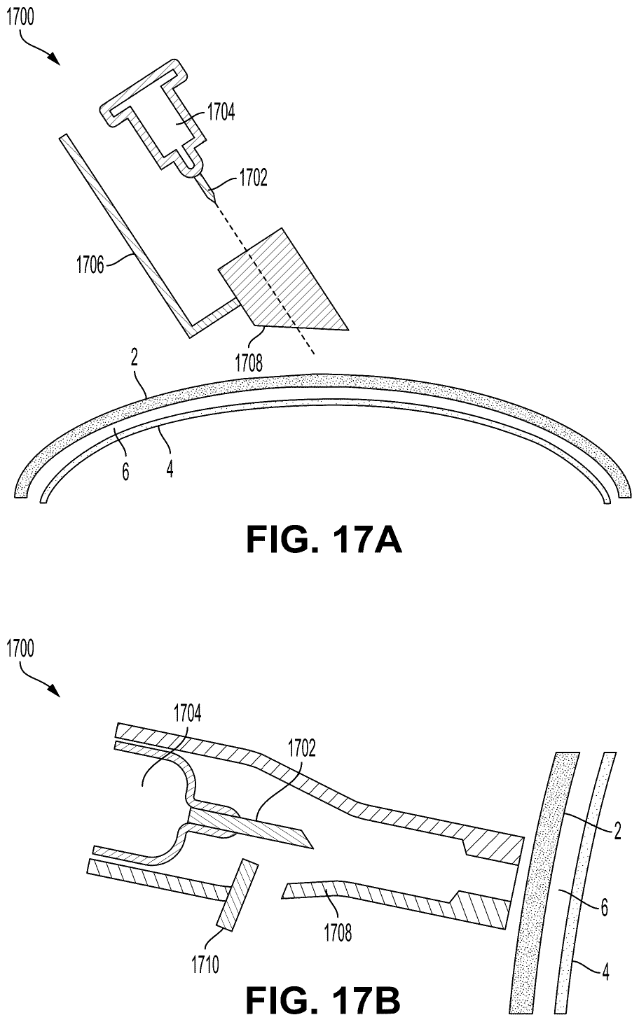

FIGS. 17A and 17B depict an exemplary drug delivery device 1700 for delivering a drug to ocular tissue. Drug delivery device 1700 may include a guide 1708 and in some embodiments, guide 1708 may include a handle 1706. Guide 1708 may be configured to be placed against the sclera 2 of a patient's eye and may define an angle and/or depth of insertion of a needle. For example, guide 1708 may first be placed against a patient's eye in a desired position. A syringe 1704 and/or a needle 1702 may then be inserted into guide 1708 and advanced within guide 1708 such that needle 1702 penetrates sclera 2. Guide 1708 may inhibit needle 1702 from being inserted further once needle 1702 has penetrated to a desirable depth within the eye, such as to the SCS 6.

As shown in FIG. 17B, guide 1708 may include a lock 1710 to selectively secure syringe 1704 and/or needle 1702 within the guide. A switch may be used to toggle the lock 1710 between securing and releasing syringe 1704 and/or needle 1702. In some embodiments, a force applied to needle 1702 during insertion may be detected. When the force decreases below a certain threshold, entry of needle 1702 into a space such as the SCS 6 may be indicated and the device may cease advancing needle 1702. In some embodiments, feedback may be provided to the user upon insertion of needle 1702 to particular depth(s). For example, the feedback may include haptic, audible, visual, or any other suitable type of feedback. In some embodiments, needle 1702 may be automatically inserted and retracted by electromechanical means. In some embodiments, the injection may similarly be automatically caused by electromechanical means upon insertion of the needle to the desired depth.

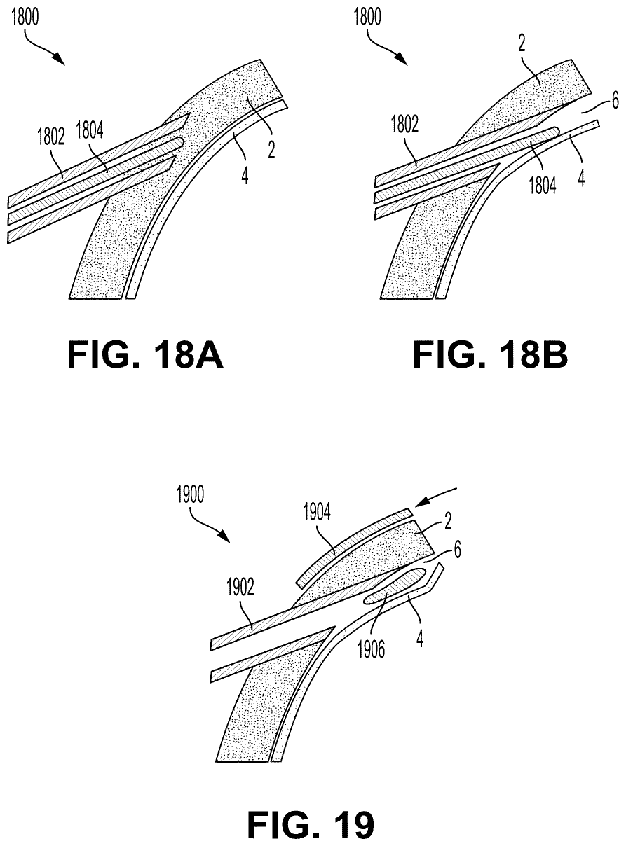

FIGS. 18A and 18B depict an exemplary drug delivery device 1800 for delivering a drug to ocular tissue. Drug delivery device 1800 may include a needle 1802 and a probe sensor 1804. Probe sensor 1804 may be initially positioned within needle 1802 and may be configured to extend outwardly from a distal end of needle 1802. In some embodiments, probe sensor 1804 may be urged outwardly of needle 1802 by a spring, motor, or another suitable mechanism. In some embodiments, probe sensor 1804 may be a capacitance sensor or a conductivity sensor, for example.

When needle 1802 is inserted into a patient's eye, probe sensor 1804 may contact tissue of the eye and detect capacitance and/or conductivity of the tissue. Upon detection of a capacitance and/or conductivity indicating that probe sensor 1804 is in contact with a choroid 4, as opposed to the sclera 2, for example, probe sensor 1804 may extend from needle 1802. Such extension may inhibit needle 1802 from being inserted further into the eye. The extension of probe sensor 1804 may further create space between the sclera 2 and choroid 4 for a drug to be injected. In some embodiments, contact of probe sensor 1804 with the choroid 4 may cause advancement of the needle 1802 to automatically cease. In some embodiments, an indication of such contact may be provided to the user.

FIG. 19 depicts exemplary drug delivery device 1900 for delivering a drug to ocular tissue. Drug delivery device 1900 may include a needle 1902 and a capacitance electrode 1904. Once needle 1902 has penetrated sclera 2 and a drug 1906 has been injected into the SCS 6, capacitance electrode 1904 may be used to detect a distribution profile of the drug 1906 in the SCS 6. For example, the capacitance electrode 1904 may detect conductivity of drug 1906. In some embodiments, saline may be added to drug 1906 to enhance conductivity. Based on values detected by the capacitance electrode 1904, a user may be able to determine whether drug 1906 has been adequately dispersed within the SCS 6.

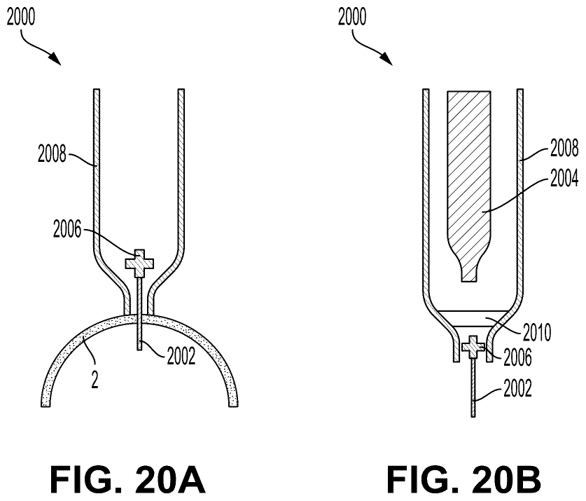

FIGS. 20A and 20B depict an exemplary drug delivery device 2000 for delivering a drug to ocular tissue. Drug delivery device 2000 may include a needle 2002 provided within a guide 2008. Guide 2008 may be configured to be placed against the sclera 2 of a patient's eye. Needle 2002 may be coupled to a gear 2006 for extending needle 2002 into the eye. In some embodiments, a syringe 2004 filled with a drug may be positioned within guide 2008. A slot 2010 may be positioned between needle 2002 and syringe 2004. Slot 2010 may be closed until needle 2002 is in a desirable position for drug delivery, thereby preventing fluid communication between syringe 2004 and needle 2002. When needle 2002 is placed into a desirable position for drug delivery, slot 2010 may then open to allow fluid communication between syringe 2004 and needle 2002 for drug delivery to the eye.

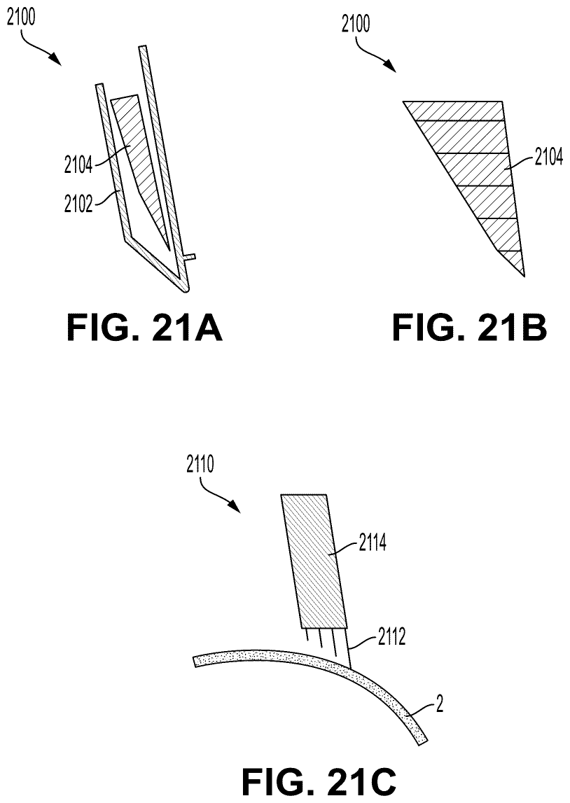

FIGS. 21A and 21B depict an exemplary drug delivery device 2100 for delivering a drug to ocular tissue. Drug delivery device 2100 may include a needle 2102 formed of a non-conductive material and having a conductive overlay 2104 thereon. In some embodiments, conductive overlay 2104 may be generally triangular. An electrical signal generated by conductive overlay 2104 may be measured and may provide an indication of a depth of insertion of the needle. Upon achieving a desired depth of insertion, advancing of needle 2102 may be automatically ceased and/or an indication may be provided to the user. In some embodiments, as shown in FIG. 21C, a device 2110 may include multiple needles 2112 each extending different lengths from a needle hub 2114. Electrical signals from each needle 2112 may be used to determine a depth of insertion of the device into the eye.

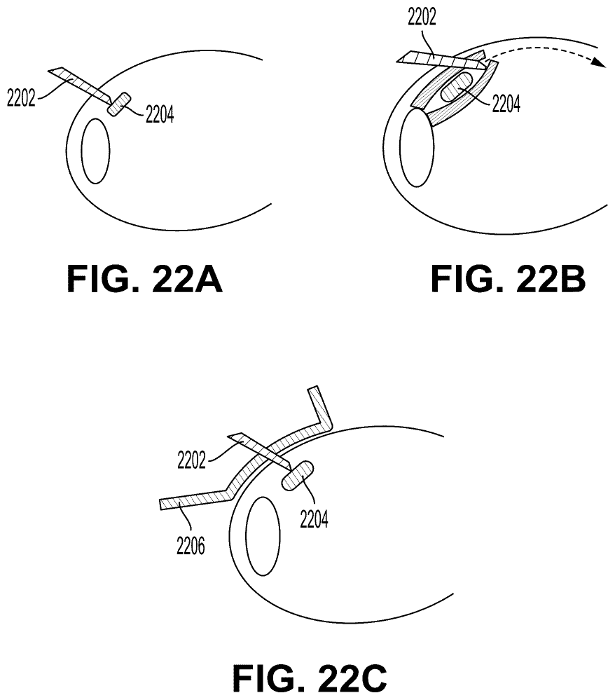

FIGS. 22A, 22B, and 22C depict still further exemplary drug delivery devices and techniques for delivering a drug to ocular tissue. In some embodiments, a plurality of injections may be performed to promote accurate drug delivery to a desirable location within the eye, such as to the SCS. First, needle 2202 may be inserted to the desired location within the eye, as shown in FIG. 22A. When needle 2202 is believed to be in the desired location, a first fluid 2204 may be injected. In some embodiments, first fluid 2204 may include a colored dye that is visible from outside the eye. In some embodiments, first fluid 2204 may be viscous such that upon injection it forms a bulb and spreads layers of the eye apart to create space for a drug.

Subsequently, a second injection may be performed, as shown in FIG. 22B. With first fluid 2204 already injected, layers of the eye may be separated, thereby creating space for the second injection and promoting dispersion of the section injection throughout the eye. The second injection may be performed using the same needle 2202 maintained in the same position, or using a different needle. During the second injection, a drug may be injected into the bulb and/or space formed by the first fluid 2204.

In some embodiments, a guide 2206 may be included to guide needle 2202 into the eye at a desired angle, as shown in FIG. 22C. Guide 2206 may include a curved surface configured to be placed against an outer surface of an eye. Guide 2206 may further include a slot configured to receive needle 2202 and angled relative to the curved surface. During an injection procedure, needle 2202 may be placed through the slot to penetrate the eye at the desired angle.

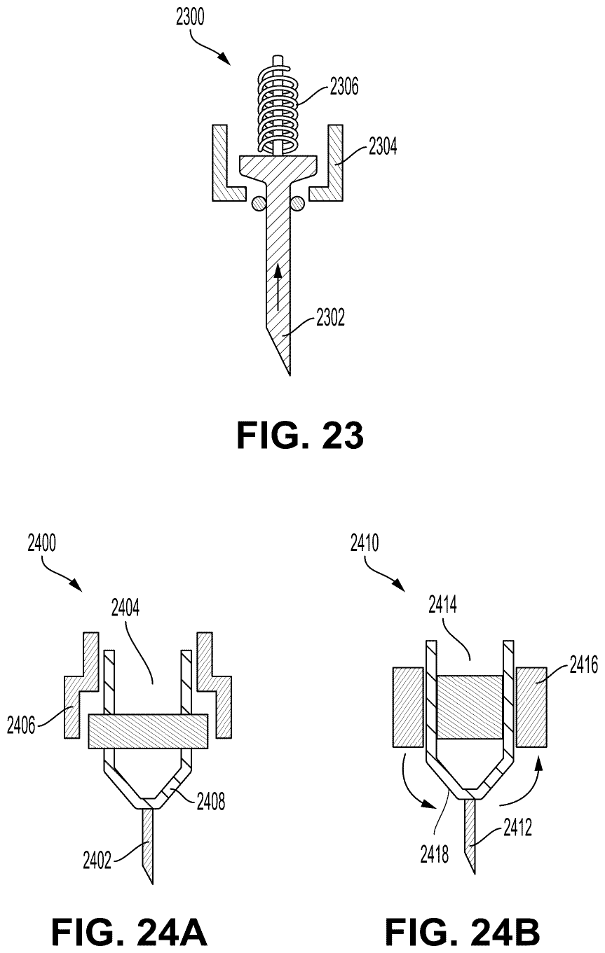

FIG. 23 depicts an exemplary drug delivery device 2300 for delivering a drug to ocular tissue. Drug delivery device 2300 may include a needle 2302, a guide 2304, and a pressure measuring device 2306. Pressure measuring device 2306 may be a mechanical device or an electromechanical device. In use, needle 2302 may be inserted into an eye to a desired position. A user may then test a pressure of the eye at the distal end of needle 2302 using pressure measuring device 2306. If the measured pressure is at an expected level for a desired region of the eye to receive a drug injection, such as the SCS, the user may then proceed to inject the drug through needle 2302.

FIGS. 24A and 24B depict exemplary drug delivery devices 2400 and 2410 for delivering a drug to ocular tissue. Device 2400 may include a syringe 2404 and an extendable needle 2402. Syringe 2404 may include a distal surface 2408 configured to be placed against a patient's eye. Device 2400 may further include a mechanism for adjusting a length of the needle for penetration. In some embodiments, the mechanism may include a movable shroud 2406 positioned around the syringe. Movement of shroud 2406 in an axial direction may cause the needle to extend and/or retract.

In place of shroud 2406, drug delivery device 2410 may include a rotating dial 2416 positioned on syringe 2414, as shown in FIG. 24B. Syringe 2414 may include a distal surface 2418 configured to be placed against a patient's eye. Dial 2416 may be turned to extend and/or retract the syringe 2414 and/or needle 2412. For example, dial 2416 may be threaded to an exterior surface of syringe 2414. In some embodiments, the mechanisms of drug delivery devices 2400 and 2410 may cause needles 2402 and 2412 to extend and/or retract incrementally. In some embodiments, the mechanisms may cause needles 2402 and 2412 to extend and/or retract continuously.

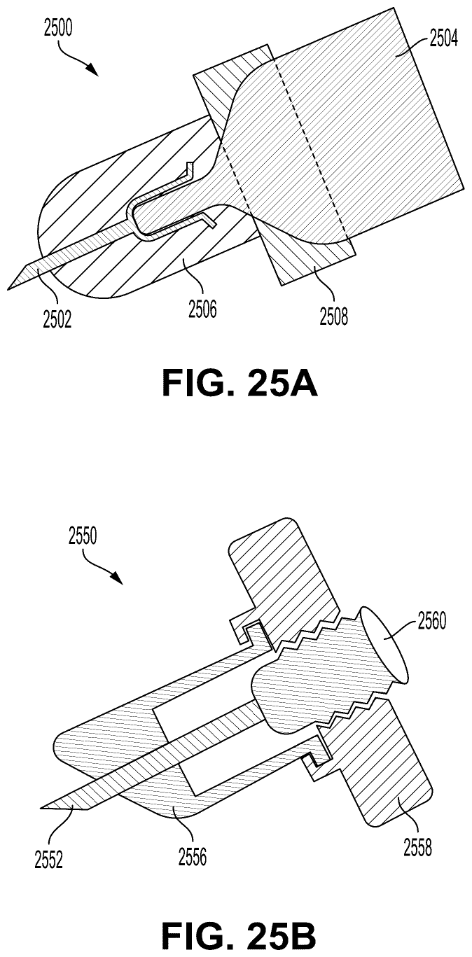

Similar to the devices shown in FIGS. 24A and 24B, the exemplary drug delivery devices 2500 and 2550 shown in FIGS. 25A and 25B may include adjustable dials configured to allow a user to adjust a length of the needle and/or a depth of insertion of the needle. For example, drug delivery device 2500 may include a needle 2502, a syringe 2504, a needle guard 2506, and a dial 2508. Needle 2502 may be a luer needle in some embodiments. Needle 2502 and syringe 2504 may be coupled together such that needle 2502 is inhibited from translating relative to syringe 2504. Dial 2508 and needle guard 2506 may be coupled together such that needle guard 2506 is inhibited from translating relative to dial 2508. To adjust a distance needle 2502 protrudes from needle guard 2506, a user may rotate dial 2508 relative to syringe 2504. The rotation may cause a change in axial position of needle guard 2506 relative to needle 2502, thereby causing needle 2502 to protrude or withdraw as desired.

Similarly, drug delivery device 2550 may include a needle 2552, a needle guard 2556, and a dial 2508. In some embodiments, needle guard 2556 may have an angled contact surface configured to be placed against an eye of a patient and configured to control an angle of insertion of needle 2552 into the eye. Needle 2552 may further include a needle hub 2560. Needle hub 2560 may be coupled to dial 2558 by a threaded interface. By virtue of the threading, rotation of dial 2558 relative to a syringe and/or needle 2552 may cause needle hub 2560 and needle 2552 to translate relative to needle guard 2556. Such rotation may thereby cause needle 2552 to protrude from or withdraw into needle guard 2556 as desired.

In some embodiments, dials 2508 and 2558 may manually or automatically advance needle 2502 or needle 2552. Needle 2502 or needle 2552 may be advanced in small increments (e.g., 50 μm) by rotation of the respective dial. Advancing needle 2502 or needle 2552 in small increments may allow for enhanced control during insertion. In some embodiments, drug delivery device 2500 or drug delivery device 2550 may include one or more sensors to detect a resistance force exerted by an eye to needle 2502 or needle 2552 as it is advanced. For example, needle 2502 or needle 2552 may experience a substantially constant resistance force as it passes through the sclera and a lower force as it reaches the SCS. Upon detection of the lower force, the device may automatically cease advancing the needle so as not to penetrate the choroid. In some embodiments, the device may provide feedback to the user indicating that needle 2502 or needle 2552 has reached the SCS.

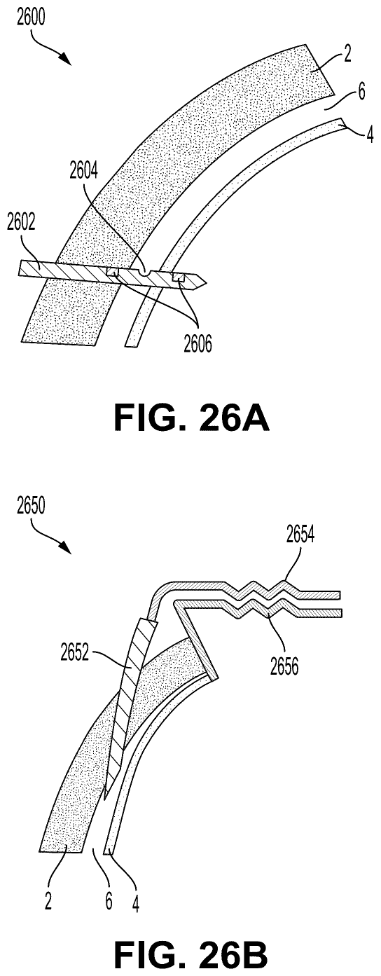

FIGS. 26A and 26B depict exemplary drug delivery devices 2600 and 2650 for delivering a drug to ocular tissue. Drug delivery device 2600, for example, may include a needle 2602 having a port 2604 positioned on a side thereof through which a drug may be dispensed. Needle 2602 may further have a plurality of sensors 2606. In some embodiments, the plurality of sensors 2606 may be capacitance sensors, and the capacitance sensors may be placed on distal and proximal sides of the port 2604. During an injection procedure, needle 2602 may be advanced into a patient's eye until a sensor 2606 on a distal side of the port 2604 indicates contact with choroid 4, and a sensor 2606 on a proximal side of the port 2604 indicates contact with the sclera 2. When sensors 2606 so indicate, a user may infer that the port 2604 is positioned in the SCS 6 and may proceed to dispense a drug through needle 2602 into the SCS 6. In some embodiments, pressure sensors may be used in lieu of, or in addition to, the capacitance sensors and may detect unique pressure characteristics of the sclera 2 and/or choroid 4.

As another example, with reference to FIG. 26B, drug delivery device 2650 may include a needle 2652, a wire 2654 connected to the needle 2652, and a ground probe 2656. Ground probe 2656 may be inserted into a patient's eye so as to contact the choroid 4. A needle 2652 may then be inserted into the patient's eye to a desired depth above the choroid 4. To ensure that the needle 2652 has not penetrated the choroid 4, a resistance between the ground probe 2656 and the needle 2652 may be measured. If the resistance indicates that the needle 2652 has not penetrated the choroid 4, the injection procedure may continue and a drug may be dispersed into the eye.

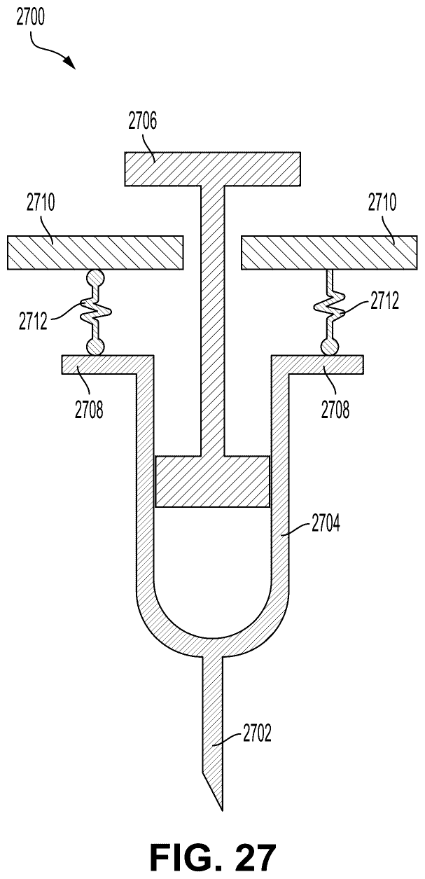

FIG. 27 depicts an exemplary drug delivery device 2700 for delivering a drug to ocular tissue. Drug delivery device 2700 may include a needle 2702, a syringe 2704, a plunger 2706, and one or more sensors 2712. Sensors 2712 may be configured to detect force and/or pressure exerted by finger flanges 2710 against shoulders 2708 of syringe 2704. During insertion of needle 2702 into an eye of a patient, the one or more sensors 2712 may detect a force and/or pressure developed between finger flanges 2710 and shoulders 2708 as the user applies pressure to the finger flanges 2710 to advance needle 2702. While the needle 2702 is passing through the sclera of the eye, the force and/or pressure detected by sensors 2712 may remain relatively constant. As the needle 2702 passes through the sclera into the SCS, the force and/or pressure may decrease significantly. Upon the drop of force and/or pressure, advancement of the needle 2702 may cease and dispersal of a drug into the SCS may proceed. In some embodiments, drug delivery device 2700 may provide feedback to the user upon the drop of force and/or pressure to alert the user to cease advancing needle 2702.

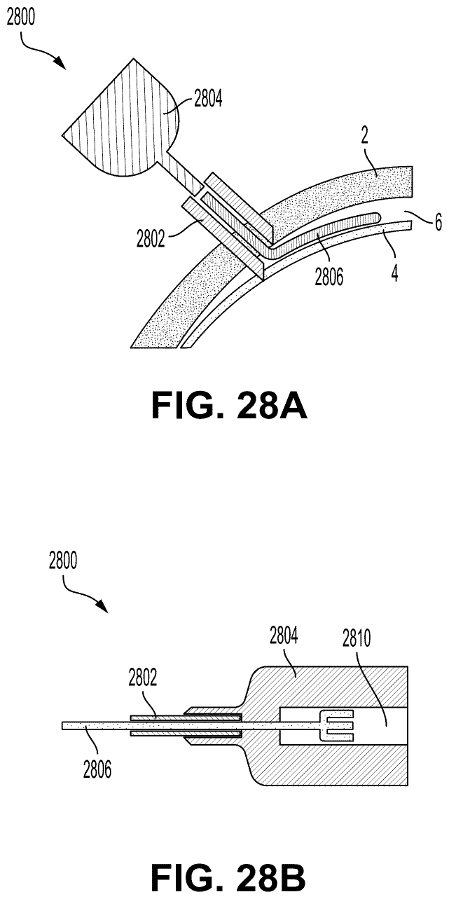

FIGS. 28A and 28B depict an exemplary drug delivery device 2800 for delivering a drug to ocular tissue. In some embodiments, drug delivery device 2800 may include a syringe 2804 and a probe 2806 configured to create space within a patient's eye for a drug. For example, as shown in FIG. 28A, during an injection procedure, a needle 2802 may be inserted into a patient's eye and through the sclera 2. The probe 2806 may then extend from needle 2802 into the SCS 6 to expand the SCS 6. Probe 2806 may be a solid material, a flexible material, or a fluid material. In some embodiments, probe 2806 may be in the form of a catheter, balloon, or stent. In some embodiments, a catheter may include a guidewire having a thickness of about 50 μm to promote positioning of the catheter in a desired layer of the eye. In some embodiments, probe 2806 may be caused to extend into the SCS 6 by a biasing mechanism such as a spring, a motor, or the like.

Extension of probe 2806 into the SCS 6 may cause the sclera 2 to move away from the choroid 4, increasing a volume of the SCS 6. Once the SCS 6 is expanded, a drug may be delivered via needle 2802. Expansion of the SCS 6 by the probe 2806 may promote dispersion of the drug through the SCS 6. In some embodiments, a force transducer may be included to detect a force exerted on needle 2802 by ocular tissue. A decrease in force exerted on needle 2802 may indicate that the SCS 6 has been appropriately expanded by probe 2806. In some embodiments, such a decrease in force may trigger drug delivery.

As shown in FIG. 28B, drug delivery device 2800 may include a mechanism 2810 for extending probe 2806 through the needle. In some embodiments, mechanism 2810 may exist within a chamber within syringe 2804. Mechanism 2810 may include any suitable biasing device, including a spring, a motor, a pump, or the like. In some examples, mechanism 2810 may function by applying a fluid pressure against a proximal end of probe 2806, thereby causing it to extend from needle 2802. A drug to be injected may be contained externally of the chamber within the syringe 2804. Alternatively, mechanism 2810 for extending probe 2806 through needle 2802 may be positioned externally of syringe 2804. For example, mechanism 2810 may be positioned at an interface between syringe 2804 and needle 2802.

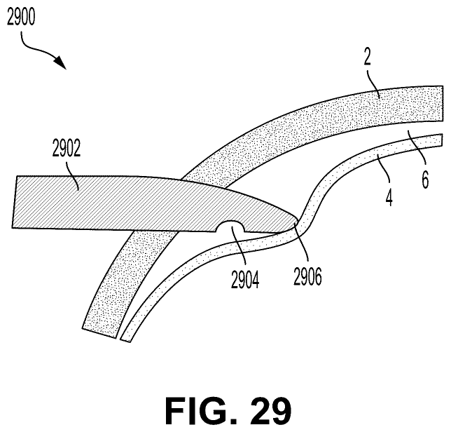

As shown in FIG. 29, a probe 2906 may be integrated with a needle 2902 in some embodiments. For example, an exemplary drug delivery device 2900 may include needle 2902 having semi-blunted distal tip that is sufficiently sharp to pierce the sclera 2 and sufficiently blunt so as not to pierce the choroid 4. Accordingly, upon insertion of needle 2902, the distal dip may pierce the sclera 2 and then displace the choroid 4 so as to increase a volume of the SCS 6. Needle 2902 may further include a port or opening 2904 on a side proximally of the distal tip through which a drug may be dispensed into the SCS 6.

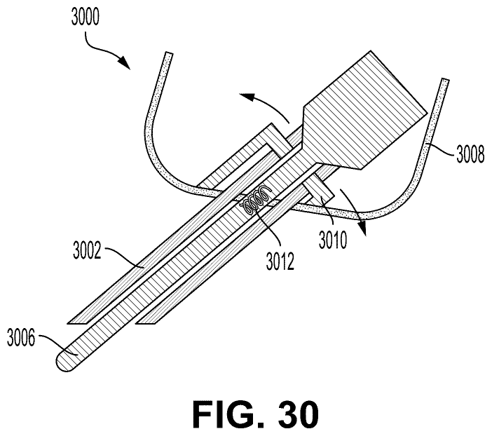

FIG. 30 depicts an exemplary drug delivery device 3000 in which a probe 3006 is used as part of a needle insertion system. Drug delivery device 3000 may include a needle 3002, probe 3006, and a guard 3008. Drug delivery device 3000 may further include anchors 3010 configured to couple needle 3002 to guard 3008. The probe 3006 may be spring loaded such that during insertion of the needle 3002 into a patient's eye and upon penetration of the sclera, the probe 3006 may extend from the needle 3002 into the SCS. As the user advances needle 3002 into a patient's eye by applying pressure to guard 3008, needle 3002 may enter the SCS in which pressure is relatively decreased. Upon entry of needle 3002 into the SCS, probe 3006 may extend into the SCS, thereby causing anchors 3010 to decouple needle 3002 from guard 3008. Once decoupled, application of force to guard 3008 no longer causes needle 3002 to advance. In some embodiments, extension of the probe 3006 may cause the needle 3002 to automatically cease advancing. In some embodiments, extension of the probe 3006 may place the needle 3002 in a configuration in which it cannot be manually advanced further. Such action may inhibit the choroid from being inadvertently penetrated by needle 3002.

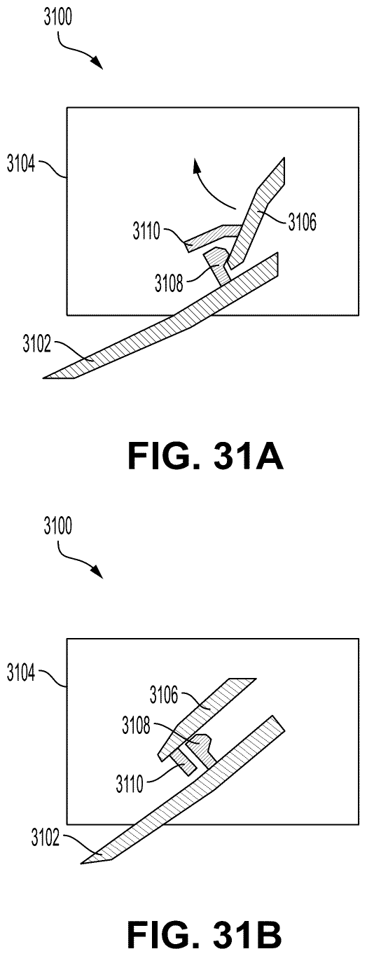

FIGS. 31A and 31B depict an exemplary drug delivery device 3100 for delivering a drug to ocular tissue. Drug delivery device 3100 may include a needle 3102, a block 3104, and a finger 3106. The needle 3102 may be coupled to the block 3104 via the finger 3106, which may be responsive to force applied to the needle 3102. For example, during insertion of needle 3102 into a patient's eye, the sclera may exert a force on needle 3102 to maintain finger 3106 in a first configuration in which finger 3106 couples needle 3102 to block 3104. The first configuration is shown in FIG. 31A. Finger 3106 may be biased so as to rotate clockwise absent interference by lug 3108, which may be fixed to needle 3102. Because needle 3102 is coupled to block 3104 by way of finger 3106 and lug 3108 in the first configuration, a user may advance needle 3102 by pushing against block 3104.

When needle 3102 advances into the SCS, the force exerted against needle 3102 may decrease substantially, allowing finger 3106 to rotate past lug 3108 and a gate 3110. In this second configuration, needle 3102 may become functionally decoupled from block 3104 such that needle 3102 no longer advances in response to pushing on the block 3104. Such action may inhibit inadvertent penetration of the choroid by the needle 3102.

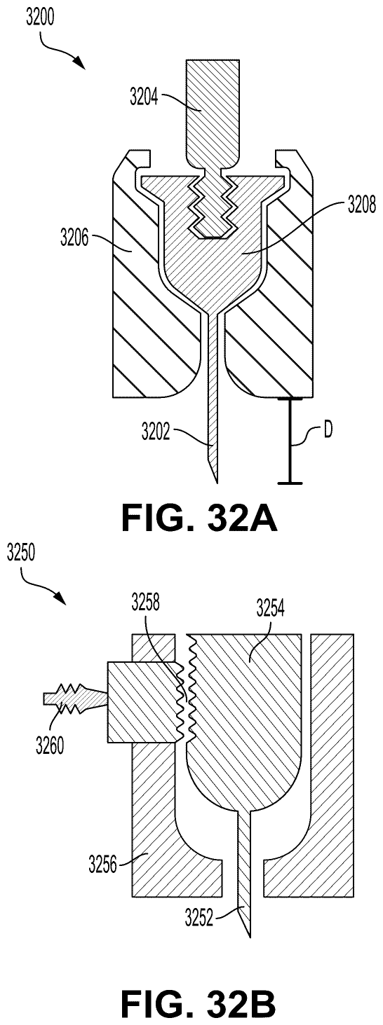

FIGS. 32A and 32B depict exemplary drug delivery devices 3200 and 3250 for delivering a drug to ocular tissue. Drug delivery device 3200 may include a needle 3202, a needle hub 3208, a syringe 3204, and a shroud 3206. Shroud 3206 may be configured to be coupled to syringe 3204, needle 3202 and/or needle hub 3208. The shroud 3206 may surround the needle 3202 such that it defines a maximum distance D the needle 3202 may be inserted into a patient's eye. Shroud 3206 may be interchangeable with shrouds of different dimensions so as to enable variation of distance D that needle 3202 may be inserted. In some embodiments, each of a plurality of shrouds may define a discrete needle length and may not be adjustable. For an injection procedure, a user may identify an appropriate needle length and may select a corresponding a shroud based on the appropriate needle length. In some embodiments, each shroud may snap on and/or off of the syringe and/or needle.

In some embodiments, the shroud may be adjustable. Drug delivery device 3250, for example, may include a needle 3252, a syringe 3254, and a shroud 3256. Shroud 3256 may be coupled to syringe 3254 via a threaded interface 3258. By rotating shroud 3256 about syringe 3254 and/or needle 3252, the threading may cause shroud 3256 to translate in an axial direction relative to needle 3252, thereby adjusting a distance that needle 3252 protrudes relative to shroud 3256. In some embodiments, shroud 3256 may include a rotatable dial configured to allow adjustment of shroud 3256 in the axial direction. In some embodiments, shroud 3206 may be coupled to syringe 3204, needle hub 3208, or needle 3202 by a set screw 3260, for example, to maintain a needle protrusion distance.

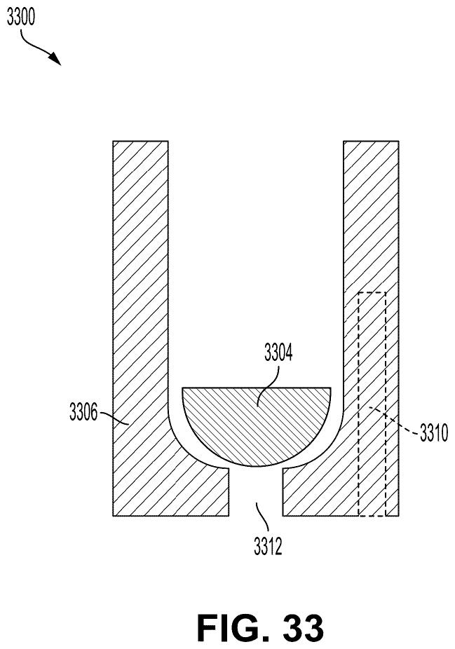

FIG. 33 depicts an exemplary drug delivery device 3300 for delivering a drug to ocular tissue. Drug delivery device 3300 may include a sleeve 3306 configured to be coupled to a syringe. In some embodiments, the sleeve 3306 may include a sensor 3310 configured to measure a thickness of a sclera of an eye. Sensor 3310 may be an air-puff sensor, a light sensor, or the like. Before inserting a syringe into sleeve 3306, the user may place sleeve 3306 against the eye to measure the sclera. After obtaining a thickness measurement for the sclera, a syringe may be mounted within sleeve 3306. Sleeve 3306 may include a stop 3304 configured to control an axial position of a syringe within sleeve 3306. An axial position of the syringe within sleeve 3306 may be adjustable such that a length of a needle projecting from sleeve 3306 through an opening 3312 is adjustable. Based on the thickness measurement, the user may select an appropriate needle length and adjust stop 3304 within sleeve 3306 accordingly so as to set an appropriate position for the syringe. The user may then place the device on the eye such that the needle penetrates the eye and sleeve 3306 is placed in contact with an outer surface of the eye. The user may then inject a drug via the needle.

Listed below are further illustrative embodiments according to the present disclosure:

(1) A medicament delivery device apparatus comprising: a needle with a sharp distalmost tip; a needle hub connected to a proximal end of the needle; and an adaptor surrounding a portion of the needle; wherein the sharp distalmost tip is configured to move from a retracted position in which the sharp distalmost tip is within the adaptor to a deployed position in which the sharp distalmost tip protrudes from the adaptor.

(2) The apparatus of (1), further comprising a user-actuated mechanism configured to selectively move the sharp distalmost tip between the retracted position and the deployed position.

(3) The apparatus of (2), further comprising a biasing member configured to urge the sharp distalmost tip toward the retracted position.

(4) The apparatus of (1), further comprising one or more sensors; and a microprocessor configured to receive signals from the one or more sensors and, based on the signals, cause the sharp distalmost tip to move from the retracted position to the deployed position.

(5) The apparatus of (4), wherein the one or more sensors include a capacitance sensor positioned on the adaptor.

(6) The apparatus of (4), wherein the one or more sensors include a pressure sensor positioned on the adaptor.

(7) A medicament delivery device apparatus comprising: a needle with a sharp distalmost tip; a needle hub connected to a proximal end of the needle; an adaptor surrounding a portion of the needle; one or more sensors; and a microprocessor configured to receive signals from the one or more sensors and, based on the signals, determine a position of the sharp distalmost tip or adaptor relative to a human organ.

(8) The apparatus of (7), wherein the one or more sensors include a capacitance sensor positioned on the adaptor.

(9) The apparatus of (7), wherein the one or more sensors include a plurality of pressure sensors positioned on the adaptor.

(10) The apparatus of (7), further comprising a microneedle; wherein the needle and the microneedle are electrically connected via a low voltage circuit.

(11) The apparatus of (7), wherein the one or more sensors include a first electrode positioned on the adaptor and the microneedle and a second electrode positioned near distalmost tip.

(12) The apparatus of (7), wherein the one or more sensors include a level configured to determine an angular position of the needle and the adaptor.

(13) The apparatus of (7), further comprising: a mechanism configured to move the sharp distalmost tip from a retracted position in which the sharp distalmost tip is positioned within the adaptor to a deployed position; wherein the microprocessor is further configured to cause, in response to determining the position of the sharp distalmost tip or adaptor, the mechanism to move the sharp distalmost tip from the retracted position to the deployed position.

(14) The apparatus of (13), wherein the microprocessor is further configured to: determine, based on the signals from the one or more sensors, that the sharp distalmost tip or adaptor has been moved out of contact with the human organ; and cause, in response to determining that the sharp distalmost tip or adaptor has been moved out of contact with the human organ, the mechanism to move the sharp distalmost tip from the deployed position to the retracted position.

(15) The apparatus of (7), wherein the one or more sensors includes a sensor configured to detect an angular position of the needle relative to a tangent of the human organ; wherein the microprocessor is further configured to determine that the angular position of the needle is a predetermined angular position.

(16) The apparatus of (15), further comprising: a mechanism configured to move the sharp distalmost tip from a retracted position in which the sharp distalmost tip is positioned within the adaptor to a deployed position; wherein the microprocessor is further configured to cause, in response to determining that the angular position of the needle is a predetermined angular position, the mechanism to move the sharp distalmost tip from the retracted position to the deployed position.

(17) The apparatus of (15), wherein the microprocessor is further configured to cause, in response to determining that the angular position of the needle is a predetermined angular position, one or more visual, audible, or tactile indications to be emitted.

(18) The apparatus of (10), wherein the microprocessor is further configured to: determine that a current of the low voltage circuit exceeds a predetermined current; and cause, in response to determining that the current of the low voltage circuit exceeds the predetermined current, one or more visual, audible, or tactile indications to be emitted.

(19) The apparatus of (7), further comprising: a first electrode positioned adjacent the sharp distalmost tip and a second electrode; wherein the microprocessor is further configured to: determine, based on a conductivity between the first electrode and second electrode, a position of the sharp distalmost tip; and cause, in response to determining position of the sharp distalmost tip, one or more visual, audible, or tactile indications to be emitted.

(20) A kit, comprising: a needle with a sharp distalmost tip; a container enclosing an ophthalmic drug; and an adaptor configured to be coupled to the needle such that the sharp distalmost tip is moveable from a retracted position in which the sharp distalmost tip is positioned within the adaptor to a deployed position in which the sharp distalmost tip protrudes from the adaptor.

(21) An apparatus for delivering a medicament to ocular tissue, the apparatus comprising: a container configured to enclose the medicament; a needle having a shaft defining a needle axis, a passage therethrough and a sharp distalmost tip, wherein the passage is configured to convey the medicament through the needle; and a needle guard at least partially surrounding the shaft of the needle, wherein the needle guard includes a distal surface, wherein the sharp distalmost tip of the needle is configured to extend through an opening in the distal surface; wherein the needle guard is moveable relative to the needle along the needle axis to control a distance from the distal surface to the distalmost tip of the needle.

(22) The apparatus of (21), wherein the distal surface is configured to be positioned against an outer surface of an eye, and the needle guard is configured to limit a depth of insertion of the needle into the eye.

(23) The apparatus of (21), further comprising: a dial coupled to one or more of the needle, the needle guard, and the container, wherein rotation of the dial is configured to cause movement of the needle guard relative to the needle.

(24) The apparatus of (23), wherein the dial is coupled to the one or more of the needle, the needle guard, and the container by a threaded connection.

(25) The apparatus of (23), wherein the rotation of the dial is configured to cause movement of the needle guard relative to the needle in discrete increments.

(26) The apparatus of (23), wherein the needle is inhibited from rotating relative to the container.

(27) The apparatus of (26), wherein the needle is keyed to the container.

(28) The apparatus of (21), further comprising: a shroud coupled to one or more of the needle, the needle guard, and the container, wherein movement of the shroud along the needle axis is configured to cause movement of the needle guard relative to the needle.

(29) The apparatus of (21), wherein the distal surface is angled relative to the needle axis.

(30) The apparatus of (21), further comprising: a sensor configured to measure a thickness of a sclera of an eye.

(31) The apparatus of (21), further comprising: a lock configured to selectively inhibit movement of the needle guard relative to the needle.

(32) An apparatus for delivering a medicament to ocular tissue, the apparatus comprising: a needle having a shaft defining a needle axis and a sharp distalmost tip; a needle guard at least partially surrounding the shaft of the needle, wherein the needle guard includes a distal surface, wherein the sharp distalmost tip of the needle is configured to extend through an opening in the distal surface, wherein the needle guard is moveable relative to the needle along the needle axis; and a dial coupled to one or more of the needle and the needle guard, wherein rotation of the dial is configured to alter a distance from the distal surface to the sharp distalmost tip.

(33) The apparatus of (32), wherein the distal surface is configured to be positioned against an outer surface of an eye and the needle guard is configured to limit a depth of insertion of the needle into the eye.

(34) The apparatus of (32), wherein the dial is coupled to the one or more of the needle and the needle guard by a threaded connection.

(35) The apparatus of (32), wherein the rotation of the dial is configured to cause movement of the needle guard relative to the needle in discrete increments.

(36) The apparatus of (32), wherein the rotation of the dial is configured to cause continuous movement of the needle guard relative to the needle.

(37) The apparatus of (32), further comprising: a sensor configured to measure a thickness of a sclera of an eye.

(38) The apparatus of (32), further comprising: a lock configured to selectively inhibit movement of the needle guard relative to the needle.

(39) A method of delivering a medicament to ocular tissue using a delivery device including a container configured to enclose the medicament, a needle having a sharp distalmost tip, and a needle guard at least partially surrounding a shaft of the needle, the method comprising: adjusting a distance from a distal surface of the needle guard to the distalmost tip of the needle; inserting, after adjusting the distance, the distalmost tip of the needle into the ocular tissue; positioning the distal surface of the needle guard against an outermost surface of the ocular tissue; and delivering a volume of the medicament to the ocular tissue via the needle.

(40) The method of (39), wherein the delivery device further includes a sensor configured to measure a thickness of a sclera, the method further comprising: measuring the thickness of the sclera using the sensor; and adjusting, based on the measured thickness, the distance from the distal surface of the needle guard to the distalmost tip of the needle.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

What is claimed is:

1. An apparatus for delivering a medicament to ocular tissue, the apparatus comprising:
a container configured to enclose the medicament;
a needle having a shaft defining a needle axis, a passage therethrough, and a sharp distalmost tip, wherein the passage is configured to convey the medicament through the needle; and
a needle guard at least partially surrounding the shaft of the needle, wherein the needle guard includes a distal surface, wherein the sharp distalmost tip of the needle is configured to extend through an opening in the distal surface;
wherein the needle guard is moveable relative to the needle along the needle axis to control a distance from the distal surface to the distalmost tip of the needle.

2. The apparatus of claim 1, wherein the distal surface is configured to be positioned against an outer surface of an eye, and the needle guard is configured to limit a depth of insertion of the needle into the eye.

3. The apparatus of claim 1, further comprising:
a dial coupled to one or more of the needle, the needle guard, and the container, wherein rotation of the dial is configured to cause movement of the needle guard relative to the needle.

4. The apparatus of claim 3, wherein the dial is coupled to the one or more of the needle, the needle guard, and the container by a threaded connection.

5. The apparatus of claim 3, wherein the rotation of the dial is configured to cause movement of the needle guard relative to the needle in discrete increments.

6. The apparatus of claim 3, wherein the needle is inhibited from rotating relative to the container.

7. The apparatus of claim 6, wherein the needle is keyed to the container.

8. The apparatus of claim 1, further comprising:
a shroud coupled to one or more of the needle, the needle guard, and the container, wherein movement of the shroud along the needle axis is configured to cause movement of the needle guard relative to the needle.

9. The apparatus of claim 1, wherein the distal surface is angled relative to the needle axis.

10. The apparatus of claim 1, further comprising:
a sensor configured to measure a thickness of a sclera of an eye.

11. The apparatus of claim 1, further comprising:
a lock configured to selectively inhibit movement of the needle guard relative to the needle.

12. An apparatus for delivering a medicament to ocular tissue, the apparatus comprising:
a needle having a shaft defining a needle axis and a sharp distalmost tip;
a needle guard at least partially surrounding the shaft of the needle, wherein the needle guard includes a distal surface, wherein the sharp distalmost tip of the needle is configured to extend through an opening in the distal surface, wherein the needle guard is moveable relative to the needle along the needle axis; and
a dial coupled to one or more of the needle and the needle guard, wherein rotation of the dial is configured to alter a distance from the distal surface to the sharp distalmost tip.

13. The apparatus of claim 12, wherein the distal surface is configured to be positioned against an outer surface of an eye, and the needle guard is configured to limit a depth of insertion of the needle into the eye.

14. The apparatus of claim 12, wherein the dial is coupled to the one or more of the needle and the needle guard by a threaded connection.

15. The apparatus of claim 12, wherein the rotation of the dial is configured to cause movement of the needle guard relative to the needle in discrete increments.

16. The apparatus of claim 12, wherein the rotation of the dial is configured to cause continuous movement of the needle guard relative to the needle.

17. The apparatus of claim 12, further comprising:
a sensor configured to measure a thickness of a sclera of an eye.

18. The apparatus of claim 12, further comprising:
a lock configured to selectively inhibit movement of the needle guard relative to the needle.

19. A method of delivering a medicament to ocular tissue using a delivery device including a container configured to enclose the medicament, a needle having a sharp distalmost tip, and a needle guard at least partially surrounding a shaft of the needle, the method comprising:
adjusting a distance from a distal surface of the needle guard to the distalmost tip of the needle;
inserting, after adjusting the distance, the distalmost tip of the needle into the ocular tissue;
positioning the distal surface of the needle guard against an outermost surface of the ocular tissue; and
delivering a volume of the medicament to the ocular tissue via the needle.

20. The method of claim 19, wherein the delivery device further includes a sensor configured to measure a thickness of a sclera, the method further comprising:
measuring the thickness of the sclera using the sensor; and
adjusting, based on the measured thickness, the distance from the distal surface of the needle guard to the distalmost tip of the needle.
